(19) United States
(12) Patent Application Publication
(10) Pub. No.: US 2025/0288517 A1
(43) Pub. Date: Sep. 18, 2025

(54) LIQUID PHARMACEUTICAL FORMULATION OF ANTI-GM-CSF ANTIBODY AND USES THEREOF

(71) Applicant: I-MAB BIOPHARMA (HANGZHOU) CO., LTD., Hangzhou (CN)

(72) Inventors: Shuyun LU, Shanghai (CN); Shicai YING, Hangzhou (CN); Zheru ZHANG, Hangzhou (CN); Junhua QIAO, Hangzhou (CN); Yihui QIN, Hangzhou (CN)

(21) Appl. No.: 19/224,048

(22) Filed: May 30, 2025

Related U.S. Application Data
(63) Continuation of application No. PCT/CN2023/135469, filed on Nov. 30, 2023.

(30) Foreign Application Priority Data
Dec. 1, 2022 (WO) PCT/CN2022/135948

(51) Int. Cl. A61K 9/08; A61K 47/12; A61K 47/22; A61K 47/26; C07K 16/24

(52) U.S. Cl. CPC A61K 9/08; A61K 47/12; A61K 47/22; A61K 47/26; C07K 16/243

(57) ABSTRACT

The present disclosure provides a liquid pharmaceutical formulation comprising an antibody which binds specifically to human GM-CSF and further excipients, and methods of treating an inflammatory or autoimmune disease or condition using the same.

### CROSS REFERENCE TO RELATED APPLICATIONS

This application is a U.S. Continuation Application of International Application No. PCT/CN2023/135469, filed on Nov. 30, 2023, which claims priority to International Application No. PCT/CN2022/135948, filed Dec. 1, 2022. The contents of each of which are hereby incorporated by reference in their entirety.

### REFERENCE TO AN ELECTRONIC SEQUENCE LISTING

The contents of the electronic sequence listing (367271.xml; Size: 23,535 bytes; and Date of Creation: May 30, 2025) is herein incorporated by reference in its entirety.

### FIELD OF THE DISCLOSURE

This disclosure relates to liquid pharmaceutical formulations comprising anti-GM-CSF antibodies.

### BACKGROUND

Granulocyte-macrophage colony-stimulating factor (GM-CSF), also known as colony stimulating factor 2 (CSF2), is a monomeric glycoprotein secreted by macrophages, T cells, mast cells, NK cells, endothelial cells and fibroblasts that functions as a cytokine. The pharmaceutical analogs of naturally occurring GM-CSF are also referred to as sargramostim and molgramostim.

GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection. GM-CSF also has some effects on mature cells of the immune system. These include, for example, inhibiting neutrophil migration and causing an alteration of the receptors expressed on the cells surface.

GM-CSF signals via signal transducer and activator of transcription, STAT5. In macrophages, it has also been shown to signal via STAT3. The cytokine activates macrophages to inhibit fungal survival. It induces deprivation in intracellular free zinc and increases production of reactive oxygen species that culminate in fungal zinc starvation and toxicity. Thus, GM-CSF facilitates development of the immune system and promotes defense against infections. GM-CSF also plays a role in embryonic development by functioning as an embryokine produced by reproductive tract.

Inhibition of GM-CSF can be useful for treating diseases such as inflammatory diseases and autoimmune disorders including rheumatoid arthritis (RA), multiple sclerosis (MS) and plaque psoriasis. Inhibition of GM-CSF can also be useful for treating cancer.

All references cited herein, including patent applications, patent publications, and UniProtKB/Swiss-Prot Accession numbers are herein incorporated by reference in their entirety, as if each individual reference were specifically and individually indicated to be incorporated by reference.

### SUMMARY OF THE DISCLOSURE

In one aspect of the present disclosure provided a liquid pharmaceutical formulation comprising
a) 20 mg/ml-200 mg/ml antibody or antigen-binding fragment thereof which binds specifically to human granulocyte macrophage colony-stimulating factor (GM-CSF) protein, wherein the antibody comprises a heavy chain variable region CDR1 (HCDR1) of SEQ ID NO: 1, a HCDR2 of SEQ ID NO: 2, a HCDR3 of SEQ ID NO: 3, a light chain variable region CDR1 (LCDR1) of SEQ ID NO: 4, a LCDR2 of SEQ ID NO: 5, and a LCDR3 of SEQ ID NO: 6;
b) 10 mM-30 mM pH buffer;
c) 130 mM-250 mM isotonicity modifier; and
d) 0.01 (w/v) %-0.03 (w/v) % surfactant,
   wherein the formulation has a pH of about 4.5 to about 7.5.

In certain embodiments, the liquid pharmaceutical formulation provided herein comprises an antibody or antigen-binding fragment thereof comprising a heavy chain variable region comprising one or more amino acid residues selected from the group consisting of:
a) Glu at position 1,
b) Arg at position 98,
c) Ser at position 72,
d) Ala at position 68,
e) Leu at position 70,
f) Ile at position 48,
g) Asp at position 26, and
h) Leu at position 29,
   according to Kabat numbering, and combinations thereof.

In certain embodiments, the heavy chain variable region comprises at least (a) Glu at position 1. In certain embodiments, the heavy chain variable region comprises a fragment of DYTLT (SEQ ID NO: 23) or GYTFT (SEQ ID NO: 24) starting at position 26 according to Kabat numbering.

In certain embodiments, the liquid pharmaceutical formulation provided herein comprises an antibody or antigen-binding fragment thereof comprising a light chain variable region comprising one or more amino acid residues selected from the group consisting of:
a) Ala at position 46,
b) Asp at position 60,
c) Asp at position 70,
d) Ser at position 43, and
e) Phe at position 87,
   according to Kabat numbering, and combinations thereof.

In certain embodiments, the antibody or antigen-binding fragment thereof provided herein comprising a heavy chain variable region comprising an amino acid sequence selected from the group consisting of SEQ ID NO: 8-17. In certain embodiments, the liquid pharmaceutical formulation provided herein comprising a light chain variable region comprising an amino acid sequence selected from the group consisting of SEQ ID NO: 19-22.

In certain embodiments, the antibody or antigen-binding fragment thereof comprises a heavy chain variable region comprises the amino acid sequence of SEQ ID NO: 14 and a light chain variable region comprises the amino acid sequence of SEQ ID NO: 22.

In certain embodiments, the antibody or antigen-binding fragment thereof further comprises a Fc region. In certain embodiments, the Fc region is of IgG1.

In certain embodiments, the pH buffer comprises citric acid, HEPES, histidine, potassium acetate, potassium citrate, potassium phosphate (KH₂PO₄), sodium acetate, sodium bicarbonate, sodium citrate, sodium phosphate (NaH₂PO₄), Tris base, and Tris-HCl.

In certain embodiments, the pH buffer comprises 20 mM histidine. In certain embodiments, the 20 mM histidine comprises about 6.2 mM histidine and about 13.8 mM histidine hydrochloride monohydrate. In certain embodiments, the pH buffer comprises about 20 mM NaAc.

In certain embodiments, the pH is ranged from about 5.5 to about 6.1. In certain embodiments, the pH is about 5.8.

In certain embodiments, the isotonicity modifier is one or more selected from the group consisting of sucrose, trehalose, mannitol, arginine, and sodium chloride. In certain embodiments, the isotonicity modifier comprises 200-220 mM sucrose. In certain embodiments, the isotonicity modifier comprises about 220 mM sucrose.

In certain embodiments, the liquid pharmaceutical formulation further comprises antioxidant, preservatives, or mixtures thereof.

In certain embodiments, the liquid pharmaceutical formulation further comprises proline and/or glycine.

In certain embodiments, the surfactant comprises polysorbate 20 or polysorbate 80. In certain embodiments, the surfactant comprises about 0.02 (w/v) % polysorbate 80.

In certain embodiments, the liquid pharmaceutical formulation comprises 50 mg/ml-150 mg/ml of the antibody. In certain embodiments, the liquid pharmaceutical formulation comprises about 100 mg/ml antibody.

In certain embodiments, the liquid pharmaceutical formulation comprises 50-150 mg/ml of the antibody, 10-20 mM histidine, 200-220 mM sucrose, and 0.01-0.03 (w/v) % polysorbate 80, and its pH is from about 5.5 to about 6.1.

In certain embodiments, the liquid pharmaceutical formulation comprises about 100 mg/ml of the antibody, about 20 mM histidine, about 220 mM sucrose, and about 0.02 (w/v) % polysorbate 80, and its pH is about 5.8.

In certain embodiments, the liquid pharmaceutical formulation is for intravenous (IV) or subcutaneous administration.

In another aspect of the present disclosure provides a method of treating an inflammatory or autoimmune disease or condition in a patient in need thereof, comprising administering to the patient the liquid pharmaceutical formulation provided herein.

In certain embodiments, the autoimmune disease is selected from the group consisting of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopeni purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

In certain embodiments, the inflammatory is selected from the group consisting of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, ulcerative colitis and COVID-19.

### DETAILED DESCRIPTION

Before describing the disclosure in detail, it is to be understood that this disclosure is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se. In case of doubt, or should there be no art recognized common understanding regarding the error range for a certain value or parameter, “about” means±1%, 2%, 3%, 4%, 5%, 6%, 7%, 8%, 9%, or 10% of this value or parameter.

It is understood that aspects and embodiments of the disclosure described herein include “comprising,” “consisting,” and “consisting essentially of aspects and embodiments.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile.

### The Anti-GM-CSF Antibody

The pharmaceutical formulations of the present disclosure may comprise an antibody, or an antigen-binding fragment thereof, that binds specifically to human GM-CSF. As used herein, the term “GM-CSF” means human granulocyte macrophage colony-stimulating factor. Antibodies to human GM-CSF are described in, for example, WO2006122797, WO2015028657, and WO2018050111.

The term “antibody”, as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody”. Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Unless specifically indicated otherwise, the term “antibody”, as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody, such as F (ab′) 2, F (ab) 2, Fab′, Fab, Fv, scFv and the like, that retain the ability to specifically bind to human GM-CSF or an epitope thereof.

An “isolated antibody”, as used herein, is intended to refer to an antibody that is substantially free of other antibodies having different antigenic specificities (e.g., an isolated antibody that specifically binds human GM-CSF is substantially free of antibodies that specifically bind antigens other than human GM-CSF).

The term “binds specifically”, or the like, means that an antibody or antigen-binding fragment thereof forms a complex with an antigen that is relatively stable under physiologic conditions. Specific binding can be characterized by a dissociation constant of at least about 1×10⁻⁸M or greater. Methods for determining whether two molecules specifically bind are well known in the art and include, for example, equilibrium dialysis, surface plasmon resonance, and the like. An isolated antibody that specifically binds human GM-CSF may, however, have cross-reactivity to other antigens, such as GM-CSF molecules from other species (orthologs). In the context of the present disclosure, multispecific (e.g., bispecific) antibodies that bind to human GM-CSF as well as one or more additional antigens are deemed to “specifically bind” human GM-CSF. Moreover, an isolated antibody may be substantially free of other cellular material or chemicals.

Exemplary anti-human GM-CSF antibodies that may be included in the pharmaceutical formulations of the present disclosure are set forth in patent application WO2018050111, the disclosure of which is incorporated by reference in its entirety.

According to certain embodiments of the present disclosure, the anti-human GM-CSF antibody, or antigen-binding fragment thereof, comprises a heavy chain complementary determining region (HCDR) 1 of SEQ ID NO: 1, an HCDR2 of SEQ ID NO: 2, and an HCDR3 of SEQ ID NO: 3. In certain embodiments, the anti-human GM-CSF antibody, or antigen-binding fragment thereof, comprises an HCVR of SEQ ID NO: 14.

According to certain embodiments of the present disclosure, the anti-human GM-CSF antibody, or antigen-binding fragment thereof, comprises a light chain complementary determining region (LCDR) 1 of SEQ ID NO: 4, an LCDR2 of SEQ ID NO: 5, and an LCDR3 of SEQ ID NO: 6. In certain embodiments, the anti-human GM-CSF antibody, or antigen-binding fragment thereof, comprises an LCVR of SEQ ID NO: 22.

In accordance with one embodiment of the present disclosure, provided is an antibody that includes the heavy chain and light chain variable domains with the CDR regions as defined in SEQ ID NO: 1-6, as shown below.

TABLE 1
Sequences of the CDR regions of 23F4

| Name | Sequences | SEQ ID NO: |
|---|---|---|
| VH CDR1 | SHYLH | 1 |
| VH CDR2 | WIFPGDDKTKYNEKFKG | 2 |
| VH CDR3 | GTKYLNWNFDV | 3 |
| VL CDR1 | KANQNVGTTLA | 4 |
| VL CDR2 | SASYRYS | 5 |
| VL CDR3 | HQYTTYPLT | 6 |

In some embodiments, an anti-GM-CSF antibody of the present disclosure includes the VH and VL CDR as listed in Table 1, with one, two or three further modifications. Such modifications can be addition, deletion or substitution of amino acids.

In some embodiments, the modification is substitution at no more than one residues from each of the CDRs. In some embodiments, the modification is substitution at one, two or three residues. In one embodiment, the modification is substitution at one of the residues. Such substitutions, in some embodiments, are conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

In some embodiments, an antibody or fragment thereof includes no more than one, no more than two, or no more than three of the above substitutions.

In some embodiments, the antibody or fragment thereof has specificity to a human GM-CSF protein and comprises a HCDR1 of SEQ ID NO: 1, a HCDR2 of SEQ ID NO: 2, a HCDR3 of SEQ ID NO: 3, a LCDR1 of SEQ ID NO: 4, a LCDR2 of SEQ ID NO: 5, and a LCDR3 of SEQ ID NO: 6. Non-limiting examples of VH are provided in SEQ ID NO:7-17, out of which SEQ ID NO: 7 is the mouse VH, and SEQ ID NO: 8-17 are humanized ones. Further, these humanized VH include one or more back-mutations to the mouse version. Likewise, non-limiting examples of VL (Vκ) are provided in SEQ ID NO: 18-22. SEQ ID NO: 18 is a mouse sequence, and SEQ ID NO: 19-22 are humanized sequences, among which SEQ ID NO: 20-22 include one or more back-mutations, as shown in the examples. The amino acid and nucleotide sequences of some of the humanized antibody 23F4 are listed in Table 2 below. The combination of the heavy chain variable regions and light chain variable regions are shown in Table 3.

TABLE 2
Humanized antibody sequences (CDR residues are underlined and
back mutations are in indicated boxes)

| | | SEQ ID |
|---|---|---|
| Name | Sequences | NO: |
| 23F4 | QVQLQQSGPELVKPGTSMKISCKTSDYTLTSHYLHWVKQRPGQGLEWIGWIFP | SEQ ID |
| VH | GDDKTKYNEKFKGKATLTSDKTSNTAYMQLSSLTSEESAVYFCARGTKYLNWN | NO. 7 |
| | FDVWGTGTTVTVSS | |
| 23F4 | EVQLVQSGAEVKKPGATVKISCKVSGYTFTSHYLHWVQQAPGKGLEWMGWIFP | SEQ ID |
| VH.1 | GDDKTKYNEKFKGRVTITADTSTDTAYMELSSLRSEDTAVYYCARGTKYLNWN | NO. 8 |
| | FDVWGQGTTVTVSS | |
| 23F4 | EVQLVQSGAEVKKPGATVKISCKVSGYTFTSHYLHWVQQAPGKGLEWMGWIFP | SEQ ID |
| VH.1a | GDDKTKYNEKFKGRVTITSDTSTDTAYMELSSLRSEDTAVYYCARGTKYLNWN | NO. 9 |
| | FDVWGQGTTVTVSS | |
| 23F4 | EVQLVQSGAEVKKPGATVKISCKVSGYTFTSHYLHWVQQAPGKGLEWMGWIFP | SEQ ID |
| VH.1b | GDDKTKYNEKFKGRATLTSDTSTDTAYMELSSLRSEDTAVYYCARGTKYLNWN | NO. 10 |
| | FDVWGQGTTVTVSS | |
| 23F4 | EVQLVQSGAEVKKPGATVKISCKVSGYTFTSHYLHWVQQAPGKGLEWIGWIFP | SEQ ID |
| VH.1c | GDDKTKYNEKFKGRATLTSDTSTDTAYMELSSLRSEDTAVYYCARGTKYLNWN | NO. 11 |
| | FDVWGQGTTVTVSS | |
| 23F4 | EVQLVQSGAEVKKPGATVKISCKVSDYTLTSHYLHWVQQAPGKGLEWIGWIFP | SEQ ID |
| VH.1d | GDDKTKYNEKFKGRATLTSDTSTDTAYMELSSLRSEDTAVYYCARGTKYLNWN | NO. 12 |
| | FDVWGQGTTVTVSS | |
| 23F4 | EVQLVQSGAEVKKPGASVKVSCKASGYTFTSHYLHWVRQAPGQGLEWMGWIFP | SEQ ID |
| VH.2 | GDDKTKYNEKFKGRVTMTRDTSISTAYMELSRLRSDDTAVYYCARGTKYLNWN | NO. 13 |
| | FDVWGQGTTVTVSS | |
| 23F4 | EVQLVQSGAEVKKPGASVKVSCKASGYTFTSHYLHWVRQAPGQGLEWMGWIFP | SEQ ID |
| VH.2a | GDDKTKYNEKFKGRVTMTSDTSISTAYMELSRLRSDDTAVYYCARGTKYLNWN | NO. 14 |
| | FDVWGQGTTVTVSS | |
| 23F4 | EVQLVQSGAEVKKPGASVKVSCKASGYTFTSHYLHWVRQAPGQGLEWMGWIFP | SEQ ID |
| VH.2b | GDDKTKYNEKFKGRVTLTSDTSISTAYMELSRLRSDDTAVYYCARGTKYLNWN | NO. 15 |
| | FDVWGQGTTVTVSS | |
| 23F4 | EVQLVQSGAEVKKPGASVKVSCKASGYTFTSHYLHWVRQAPGQGLEWIGWIFP | SEQ ID |
| VH.2c | GDDKTKYNEKFKGRATLTSDTSISTAYMELSRLRSDDTAVYYCARGTKYLNWN | NO. 16 |
| | FDVWGQGTTVTVSS | |
| 23F4 | EVQLVQSGAEVKKPGASVKVSCKASDYTLTSHYLHWVRQAPGQGLEWIGWIFP | SEQ ID |
| VH.2d | GDDKTKYNEKFKGRATLTSDTSISTAYMELSRLRSDDTAVYYCARGTKYLNWN | NO. 17 |
| | FDVWGQGTTVTVSS | |
| 23F4 | DIVLTQPQKFLSTSVGDRVSVTCKANQNVGTTLAWYQQKPGQSPKALIYSASY | SEQ ID |
| Vk | RYSGVPDRFTGSGSGTDFTLTISNVQSEDLAEYFCHQYTTYPLTFGGGTKLEI | NO. 18 |
| | K | |
| 23F4 | DIQLTQSPSFLSASVGDRVTITCKANQNVGTTLAWYQQKPGKAPKLLIYSASY | SEQ ID |
| Vk.1 | RYSGVPSRFSGSGSGTEFTLTISSLQPEDFATYYCHQYTTYPLTFGGGTKVEI | NO. 19 |
| | K | |
| 23F4 | DIQLTQSPSFLSASVGDRVTITCKANQNVGTTLAWYQQKPGKAPKALIYSASY | SEQ ID |
| Vk.1a | RYSGVPSRFSGSGSGTEFTLTISSLQPEDFATYYCHQYTTYPLTFGGGTKVEI | NO. 20 |
| | K | |
| 23F4 | DIQLTQSPSFLSASVGDRVTITCKANQNVGTTLAWYQQKPGKAPKALIYSASY | SEQ ID |
| Vk.1b | RYSGVPDRFSGSGSGTDFTLTISSLQPEDFATYYCHQYTTYPLTFGGGTKVEI | NO. 21 |
| | K | |
| 23F4 | DIQLTQSPSFLSASVGDRVTITCKANQNVGTTLAWYQQKPGKSPKALIYSASY | SEQ ID |
| Vk.1c | RYSGVPDRFSGSGSGTDFTLTISSLQPEDFATYFCHQYTTYPLTFGGGTKVEI | NO. 22 |
| | K | |

TABLE 3
Combination of VH/Vκ for Humanization Antibody 23F4

| | Vκ | | | | |
|---|---|---|---|---|---|
| | 23F4 | 23F4 | 23F4 | 23F4 | 23F4 |
| VH | Vk.1 | Vk.1a | Vk.1b | Vk.1c | Vk |
| 23F4 | Hu23F4-1 | Hu23F4-2 | Hu23F4-3 | Hu23F4-4 | |
| VH.1 | | | | | |
| 23F4 | Hu23F4-5 | Hu23F4-6 | Hu23F4-7 | Hu23F4-8 | |
| VH.1a | | | | | |
| 23F4 | Hu23F4-9 | Hu23F4-10 | Hu23F4-11 | Hu23F4- | |
| VH.1b | | | | 12 | |
| 23F4 | Hu23F4-13 | Hu23F4-14 | Hu23F4-15 | Hu23F4- | |
| VH.1c | | | | 16 | |
| 23F4 | Hu23F4-17 | Hu23F4-18 | Hu23F4-19 | | |
| VH.1d | | | | | |
| 23F4 | Hu23F4-20 | Hu23F4-21 | Hu23F4-22 | Hu23F4- | |
| VH.2 | | | | 23 | |
| 23F4 | Hu23F4-24 | Hu23F4-25 | Hu23F4-26 | Hu23F4- | |
| VH.2a | | | | 27 | |
| 23F4 | Hu23F4-28 | Hu23F4-29 | Hu23F4-30 | Hu23F4- | |
| VH.2b | | | | 31 | |
| 23F4 | Hu23F4-32 | Hu23F4-33 | Hu23F4-34 | Hu23F4- | |
| VH.2c | | | | 35 | |
| 23F4 | Hu23F4-36 | Hu23F4-37 | Hu23F4-38 | | |
| VH.2d | | | | | |
| 23F4 | | | | | 23F4 |
| VH | | | | | chimera |

The back-mutations are shown to be useful for retaining certain characteristics of the anti-GM-CSF antibodies. Accordingly, in some embodiments, the anti-GM-CSF antibodies of the present disclosure, in particular the human or humanized ones, include one or more of the back-mutations. In some embodiments, the VH back-mutation (i.e., included amino acid at the specified position) is one or more selected from (a) Glu at position 1 (E1), (b) Arg at position 98 (R98), (c) Ser at position 72 (S72), (d) Ala at position 68 (A68), (e) Leu at position 70 (L70), (f) Ile at position 48 (148), (g) Asp at position 26 (D26), and (h) Leu at position 29 (L29), according to Kabat numbering, and combinations thereof.

In some embodiments, the humanized antibody includes at least VH back-mutation E1. In some embodiments, the humanized antibody includes at least VH back-mutations E1 and R98. In some embodiments, the humanized antibody includes at least VH back-mutations E1 and another as listed above. In some embodiments, the humanized antibody includes at least VH back-mutation group (E1, R98 and S72), (E1, R98, S72 and A68), (E1, R98, S72, A68, L70 and 148), (E1, R98, S72, A68, L70, 148, D26 and L29), (E1 and S72), (E1, S72 and L70), (E1, S72, L70, 148 and A68), (E1, S72, L70, 148, A68, D26 and L29).

In some embodiments, the heavy chain variable region comprises a fragment of DYTLT (SEQ ID NO: 23) or GYTFT (SEQ ID NO: 24) at the N-terminal end of the CDR1, i.e., starting at position 26 according to Kabat numbering. In one embodiment, the heavy chain variable region comprises DYTLT (SEQ ID NO: 23). In one embodiment, the heavy chain variable region comprises GYTFT (SEQ ID NO: 24).

In some embodiments, the humanized antibody includes one or more of the back-mutations. In some embodiments, the VL back-mutation is one or more selected from (a) Ala at position 46 (A46), (b) Asp at position 60 (D60), (c) Asp at position 70 (D70), (d) Ser at position 43 (S43), and (f) Phe at position 87 (F87), according to Kabat numbering, and combinations thereof.

In some embodiments, the humanized antibody includes at least two, three or four of VL back-mutations A46, D60, D70, S43, or F87. In some embodiments, the humanized antibody includes at least VL back-mutation A46. In some embodiments, the humanized antibody includes at least VL back-mutations A46 and D60 and another as listed above. In some embodiments, the humanized antibody includes at least VL back-mutation group (A46, D60 and D70) or (A46, D60, D70, S43 and F87).

In some embodiments, the humanized antibody includes at least VH back-mutations (E1, R98, S72, A68, L70 and 148) and no VL back-mutations. In some embodiments, the humanized antibody includes at least VH back-mutations (E1, S72, L70, 148, A68, D26 and L29) and no VL back-mutations. In some embodiments, the humanized antibody includes at least VH back-mutations (E1 and S72) and VL back-mutations (A46, D60, D70, S43 and F87).

In some embodiments, the anti-GM-CSF antibody of the present disclosure includes a VH of SEQ ID NO: 8-17, and a VL of SEQ ID NO: 19-22, or their respective biological equivalents. A biological equivalent of a VH or VL is a sequence that includes the designated amino acids while having an overall 80%, 85%, 90%, 95%, 98% or 99% sequence identity. A biological equivalent of SEQ ID NO: 10, therefore, can be a VH that has an overall 80%, 85%, 90%, 95%, 98% or 99% sequence identity to SEQ ID NO: 10 but retains the CDRs (SEQ ID NO: 1-3 or their variants), and optionally retains one or more, or all of the back-mutations.

In one embodiment, the VH has the amino acid sequence of SEQ ID NO: 11 and the VL has the amino acid sequence of SEQ ID NO: 19. In one embodiment, the VH has the amino acid sequence of SEQ ID NO: 17 and the VL has the amino acid sequence of SEQ ID NO: 19. In one embodiment, the VH has the amino acid sequence of SEQ ID NO: 11 and the VL has the amino acid sequence of SEQ ID NO: 22. Each of the recited sequences, it is noted, can also be substituted with their biological equivalents.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody provided herein further comprises a heavy chain constant region, a light chain constant region, an Fc region, or the combination thereof.

The Fc region can be engineered to enhance or eliminate effector function. IgG antibodies can induce direct anti-tumor effects by way of indirect anti-tumor effects via the Fc-mediated effector functions that engage other immune cells or killer mechanisms. “Effector functions” or “antibody effector functions” as used herein refer to biological activities attributable to the binding of Fc region of an antibody to its effectors such as C1 complex and Fc receptor (FcγRIIa or FcγRIIIa). Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and antibody dependent cell mediated phagocytosis (ADCP), where nonspecific cytotoxic cells that express Fcγ receptors (FcγRs) recognize bound antibody on a target cell and subsequently cause phagocytosis of the target cell.

Among the four IgG subclasses, IgG1 and IgG3 induce the strongest Fc-effector functions. However, since IgG1 has the longest half-life and is more stable than IgG3, most therapeutic antibodies with Fc-mediated functions are of IgG1 isotype.

IgG2 and IgG4 isotypes have significantly lower binding affinity to FcγRs. Recent evidence suggests that the IgG2 isotype is not completely devoid of effector function, whereas the IgG4 isotype can undergo in vivo Fab arm exchange leading to bispecific antibody and off-target effects.

In certain embodiments, the isotype of the antibody provided herein is IgG1. In certain embodiments, the isotype of the antibody is human IgG1.

The antibodies and fragments of the present disclosure can be mono-specific or bispecific antibodies or fragments, in some embodiments. For a bispecific antibody, the other specificity can be to a different target epitope of GM-CSF or a different target protein which is useful for a particular use, e.g., therapeutic use. In one aspect, the target protein is s cytokine such as TNF-alpha, IL-6, IL-1, and IL-17. In another aspect, the target protein is a chemokine, such as CCL2, CXCL12, and CXCL13. In another aspect, the target protein is a cell surface protein, such as CD3, CSF-1R, CD20, and CD73.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated or connected by other means to another molecule to form a bi-functional molecule. The second molecule may be one of therapeutic agents, prodrugs, peptides, proteins, enzymes, viruses, lipids, biological response modifiers, pharmaceutical agents, or PEG. Some non-limiting examples are cytokines or other soluble factors, such as IL-10, IL-25, IL-27, IL-33, IL-35, and IL-36. Also provided, in some embodiments, are antibody-drug conjugates which include an antibody or fragment of the present disclosure and a small molecule drug.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

### The Pharmaceutical Formulations

The antibody which is formulated is preferably essentially pure and desirably essentially homogeneous (e.g., free from contaminating proteins etc.). “Essentially pure” antibody means a composition comprising at least about 90% by weight of the antibody, based on total weight of proteins in the composition, preferably at least about 95% by weight, “Essentially homogeneous” antibody means a composition comprising at least about 99% by weight of antibody, based on total weight of proteins in the composition.

The term “pharmaceutical formulation” refers to a preparation that contains an anti-GM-CSF antibody in such form as to permit the biological activity of the antibody to be effective, and which contains no additional components which are unacceptably toxic to a subject to which the formulation would be administered.

The formulation can be a liquid. Liquid formulations are aqueous solutions or suspensions, prepared in a suitable aqueous solvent, such as water or an aqueous/organic mixture, such as water alcohol mixtures.

In certain embodiments, the liquid pharmaceutical formulation comprises 20 mg/ml-200 mg/ml antibody, e.g. 30 mg/ml-200 mg/ml, 40 mg/ml-200 mg/ml, 70 mg/ml-200 mg/ml, 80 mg/ml-200 mg/ml, 50 mg/ml-200 mg/ml, 60 mg/ml-200 mg/ml, 90 mg/ml-200 mg/ml, 100 mg/ml-200 mg/ml, 110 mg/ml-200 mg/ml, 120 mg/ml-200 mg/ml, 130 mg/ml-200 mg/ml, 140 mg/ml-200 mg/ml, 150 mg/ml-200 mg/ml, 160 mg/ml-200 mg/ml, 170 mg/ml-200 mg/ml, 180 mg/ml-200 mg/ml, 190 mg/ml-200 mg/ml, 20 mg/ml-190 mg/ml, 20 mg/ml-180 mg/ml, 20 mg/ml-170 mg/ml, 20 mg/ml-160 mg/ml, 20 mg/ml-150 mg/ml, 20 mg/ml-120 mg/ml, 20 mg/ml-110 mg/ml, 20 mg/ml-140 mg/ml, 20 mg/ml-130 mg/ml, 20 mg/ml-100 mg/ml, 20 mg/ml-90 mg/ml, 20 mg/ml-80 mg/ml, 20 mg/ml-70 mg/ml, 20 mg/ml-60 mg/ml, 20 mg/ml-50 mg/ml, 20 mg/ml-40 mg/ml, 30 mg/ml-190 mg/ml, 40 mg/ml-180 mg/ml, 50 mg/ml-170 mg/ml, 60 mg/ml-160 mg/ml, 70 mg/ml-150 mg/ml, 80 mg/ml-140 mg/ml, 90 mg/ml-130 mg/ml, 100 mg/ml-120 mg/ml. In certain embodiments, the liquid pharmaceutical formulation comprises 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, or 200 mg/ml antibody.

In another embodiment the pharmaceutical formulation further comprises additional excipients. The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity.

“Excipients” includes, but is not limited to, stabilizers, for example, human serum albumin (HSA), bovine serum albumin (BSA), α-casein, globulins, α-lactalbumin, LDH, lysozyme, myoglobin, ovalbumin, RNase A; buffering agents, for example, citric acid, HEPES, PBS, histidine, potassium acetate, potassium citrate, potassium phosphate (KH₂PO₄), sodium acetate, sodium bicarbonate, sodium citrate, sodium phosphate (NaH₂PO₄), Tris base, and Tris-HCl; amino acids/metabolites, for example, glycine, alanine (α-alanine, β-alanine), arginine, betaine, leucine, lysine, glutamic acid, aspartic acid, histidine, proline, 4-hydroxyproline, sarcosine, γ-aminobutyric acid (GABA), opines (alanopine, octopine, strombine), and trimethylamine N-oxide (TMAO); surfactants, for example, polysorbate 20 and 80, and poloxamer 407; lipid molecules, for example, phosphatidyl choline, ethanolamine, and acethyltryptophanate; polymers, for example, polyethylene glycol (PEG), and polyvinylpyrrolidone (PVP) 10, 24, 40; low molecular weight excipients, for example, arabinose, cellobiose, ethylene glycol, fructose, fucose, galactose, glycerin/glycerol, glucose, inositol, lactose, maltose, maltotriose, mannose, melibiose, 2-methyl-2,4-pentanediol, octulose, propylene glycol, raffinose, ribose, sucrose, trehalose, xylitol, and xylose; and high molecular weight excipients, for example, cellulose, β-cyclodextrin, dextran (10 kd), dextran (40 kd), dextran (70 kd), ficoll, gelatin, hydroxypropylmethyl-cellulose, hydroxyethyl starch, maltodextrin, methocel, PEG (6 kd), poly dextrose, polyvinylpyrrolidone (PVP) k15 (10 kd), PVP (40 kd), PVP k30 (40 kd), PVP k90 (1000 kd), sephadex G-200, and starch; antioxidants, for example, ascorbic acid, cysteine HCl, thioglycerol, thioglycolic acid, thiosorbitol, and glutathione; reducing agents, for example, cysteine HCl, dithiothreotol, and other thiol or thiophenes; chelating agents, for example, EDTA, EGTA, glutamic acid, and aspartic acid; inorganic salts/metals, for example, Ca²⁺, Ni²⁺, Mg²⁺, Mn²⁺, Na₂SO₄, (NH₄)₂SO₄, Na₂HPO₄/NaH₂PO₄, K₂HPO₄/KH₂PO₄, MgSO₄, and NaF; organic salts, for example, Na acetate, Na polyethylene, Na caprylate (Na octanoate), proprionate, lactate, succinate, and citrate; organic solvents, for example, acetonitrile, dimethylsulfoxide (DMSO), and ethanol. For additional information regarding excipients, see Remington's Pharmaceutical Sciences (by Joseph P. Remington, 18th ed., Mack Publishing Co., Easton. Pa.), which is incorporated herein in its entirety.

By “isotonic” is meant that the formulation of interest has essentially the same osmotic pressure as human blood. Isotonic formulations generally have an osmotic pressure from about 250 to about 350 mOsm (e.g. from about 250 to about 340, from about 250 to about 330, from about 250 to about 320, from about 250 to about 310, from about 250 to about 300, from about 260 to about 350, from about 270 to about 350, from about 280 to about 350, from about 290 to about 350, from about 300 to about 350, from about 260 to about 340, from about 270 to about 330, from about 280 to about 320, from about 290 to about 310 mOsm). Isotonicity can be measured using a vapor pressure or ice-freezing type osmometer, for example. In certain embodiments, the isotonicity is about 300 mOsm. A isotonicity modifier can be one or more selected from the group consisting of sucrose, trehalose, mannitol, arginine, and sodium chloride. In certain embodiments, the isotonicity modifier is of a range of about 130-250 mM (e.g. from about 130 mM to about 240 mM, from about 150 mM to about 240 mM, from about 180 mM to about 240 mM, from about 200 mM to about 240 mM, from about 130 mM to about 220 mM, from about 150 mM to about 220 mM, from about 180 mM to about 220 mM, from about 130 mM to about 210 mM, from about 150 mM to about 210 mM, from about 180 mM to about 210 mM, from about 200 mM to about 220 mM, from about or 200 to about 210 mM). In certain embodiments, the isotonicity modifier is about 130 mM, about 140 mM, about 150 mM, about 160 mM, about 170 mM, about 180 mM, about 190 mM, about 200 mM, about 210 mM, about 220 mM, about 230 mM, about 240 mM, or about 250 mM. In certain embodiments, the isotonicity modifier comprises sucrose or trehalose.

As used herein, “pH buffer” refers to a buffered solution that resists changes in pH by the action of its acid-base conjugate components. The buffer of this disclosure preferably has a pH in the range from about 4.5 to about 7.5, preferably from about 5.0 to about 7.0, for example from about 5.0 to about 6.9, about 5.2 to about 6.8, about 5.3 to about 6.7, about 5.4 to about 6.6, about 5.5 to about 6.5, about 5.6 to about 6.4, about 5.7 to about 6.3, about 5.8 to about 6.2, about 5.9 to about 6.1, about 5.5 to about 6.4, about 5.5 to about 6.3, about 5.5 to about 6.2, about 5.5 to about 6.1, or about 5.5 to about 6.0. In one embodiment the buffer has a pH of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0. In one embodiment the buffer has a pH 5.8. Various means may be utilized in achieving the desired pH level, including, but not limited to the addition of the appropriate buffer.

In certain embodiments, the pH buffer comprises histidine, acetate, citrate, and succinate. In certain embodiments, the pH buffer comprises from about 10 to about 30 mM histidine and/or from about 10 to about 30 mM acetate, for example, from about 10 to about 25 mM, from about 10 to about 20 mM, or from about 15 mM to about 20 mM histidine and/or from about 10 to about 25 mM, from about 10 mM to about 20 mM, or from about 15 mM to about 20 mM acetate. In certain embodiments, the pH buffer comprises about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM histidine or acetate.

In certain embodiments, the pH buffer comprises L-histidine and histidine hydrochloride monohydrate. In certain embodiments, the pH buffer comprises 20 mM histidine comprising about 4.4 mM L-histidine and about 15.6 mM histidine hydrochloride monohydrate, about 5.2 mM L-histidine and about 14.8 mM histidine hydrochloride monohydrate, about 6.2 mM L-histidine and about 13.8 mM histidine hydrochloride monohydrate, about 6.8 mM L-histidine and about 13.2 mM histidine hydrochloride monohydrate, about 7.8 mM L-histidine and about 12.2 mM histidine hydrochloride monohydrate, about 8.4 mM L-histidine and about 11.6 mM histidine hydrochloride monohydrate, or about 9 mM L-histidine and about 11 mM histidine hydrochloride monohydrate.

As used herein, a “surfactant” refers to a surface-active agent, preferably a nonionic surfactant. Examples of surfactants herein include polysorbate (for example, polysorbate 20, polysorbate 40, polysorbate 60, polysorbate 65, polysorbate 80, and, polysorbate 85); poloxamer (e.g. poloxamer 188 and poloxamer 407); Triton; sodium dodecyl sulfate (SDS); sodium laurel sulfate; sodium octyi glycoside: lauryl-, myristyl-, linoleyl-, or stearyl-sulfobetaine; lauryl-, myristyl-, linoleyl- or stearyl-sarcosine: linoleyl-, myristyl-, or cetyl-betaine; lauroamidopropyl-, cocamidopropyl-, linoleamido propyl-, myristamidopropyl-, palmidopropyl-, or isostearamidopropyl-betaine (e.g. lauroamidopropyl); myristamidopropyl-, palmidopropyl-, or isostearamidopropyl-dimethylamine: sodium methyl cocoyl-, or disodium methyl oleyl-taurate; and the MONAQUAT™ series (Mona Industries, Inc., Paterson, N.J.): polyethyl glycol, polypropyl glycol, and copolymers of ethylene and propylene glycol (e.g. Piuronics, PF68 etc); etc.

The surfactant concentration is generally from about 0.0001% to about 1.0%, from about 0.01% to about 0.5%, for example, from about 0.015 (w/v) % to about 0.03 (w/v) %, from about 0.02 (w/v) % to about 0.03 (w/v) %, from about 0.025 (w/v) % to about 0.03 (w/v) %, from about 0.01 (w/v) % to about 0.025 (w/v) %, from about 0.01 (w/v) % to about 0.02 (w/v) %, or from about 0.01 (w/v) % to about 0.015 (w/v) %. In one embodiment, the surfactant provided herein comprises polysorbate 80 or polysorbate 20. In certain embodiments, the surfactant comprises about 0.01 (w/v) %, 0.015 (w/v) %, 0.02 (w/v) %, 0.025 (w/v) %, or 0.03 (w/v) % polysorbate 80 or polysorbate 20. In certain embodiments, the surfactant comprises about 0.02 (w/v) % polysorbate 80.

In certain embodiments, the liquid pharmaceutical formulation further comprises antioxidant, preservatives, or mixtures thereof.

The term “antioxidant” refers to an agent that inhibits the oxidation of other molecules. Examples of the antioxidant include ascorbic acid, citrate, lipoic acid, uric acid, cysteine HCl, monothioglycerol, thioglycerol, thioglycolic acid, thiosorbitol, tocopherol, carotene, lycopene and glutathione; reducing agents, for example, cysteine HCl, dithiothreotol, phosphonate compounds, e.g., etidronic acid, desferoxamine and malate, and other thiol or thiophenes and methionine. In other embodiments the antioxidant is a metal chelator. Metal chelators include, but are not limited to ethylenediaminetetraacetate (“EDTA”), ethylene glycol tetra acetic acid (“EGTA”), (thiamine tetrahydrofurfuryl disulfide (“TTFD”), and 2,3-dimercaptosuccinic acid (“DMSA”). In certain embodiments the formulation comprises about 1 mM to about 50 mM antioxidant. In one embodiment the formulation comprises about 5 mM, about 10 mM, about 15 mM, about 20 mM, about 25 mM, about 30 mM, about 35 mM, about 40 mM, or about 45 mM antioxidant.

The term “preservative” refers to pharmaceutically acceptable excipients which prevent the growth of micro-organisms within the composition. More particularly, the disclosure provides a preservative containing multi-dose liquid composition which protects the composition against microbial contamination.

In one embodiment, the preservative is present within the composition in an amount of between 0.001 to 2% (w/v). In one embodiment, the preservative is present within the composition in an amount of between 0.002 to 1% (w/v). In one embodiment, the one or more preservative is selected from phenol, m-cresol, benzyl alcohol, chlorobutanol, ethanol, phenoxyethanol, p-chlor-m-cresol, methyl paraben, propyl paraben, benzalkonium chloride, thiomersal or any combinations thereof. In one embodiment, the one or more preservative is selected from phenol, m-cresol, benzyl alcohol and chlorobutanol.

The viscosity of an anti-GM-CSF antibody formulation can be controlled for subcutaneous, intravenous or intramuscular administration. The viscosity can be affected by protein concentration and pH. For example, as the protein concentration increases, the viscosity can increase. An increase in pH can decrease the viscosity of the anti-GM-CSF antibody formulation. In some protein formulations, sodium chloride is added to reduce the viscosity of the formulation. Additional components that can affect viscosity of an anti-GM-CSF antibody formulation are amino acids such as histidine and arginine.

The liquid pharmaceutical formulations described herein can have various viscosities. Methods of measuring viscosity of liquid pharmaceutical formulations are known to those in the art, and can include, e.g., a rheometer (e.g., Anton Paar MCR301 Rheometer with either a 50 mm, 40 mm or 20 mm cone accessory). In some embodiments of the present disclosure, the viscosities were reported at a high shear limit of 1000 per second shear rate. In some embodiments, the liquid pharmaceutical formulations has a viscosity between 1.0 cP±10% and 20 cP±10%. In some embodiments, the liquid pharmaceutical formulations has a viscosity of less than 20 cP, less than 18 cP, less than 15 cP, less than 13 cP, or less than 11 cP. One of skill in the art will appreciate that viscosity is dependent on temperature, thus, unless otherwise specified, the viscosities provided herein are measured at 25° C. unless otherwise specified. In some embodiments, the viscosity of the liquid pharmaceutical formulations is 1.0 cP±10%, 2.0 cP±10%, 3.0 cP±10%, 3.1 cP±10%, 3.2 cP±10%, 3.5 cP±10%, 3.6 cP±10%, 3.8 cP±10%, 4.0 cP±10%, 5.0 cP±10%, 5.3 cP±10%, 6.0 cP±10%, 6.3 cP±10%, 6.4 cP±10%, 6.8 cP±10%, 7.0 cP±10%, 7.1 cP±10%, 7.4 cP±10%, 8.0 cP±10%, 9.0 cP±10%, 10.0 cP±10%, 11.0 cP±10%, 12.0 cP±10%, 13.0 cP±10%, 14.0 cP±10%, 15.0 cP±10%, or 16 cP±10% at 25° C.

In certain embodiments, the liquid pharmaceutical formulation further comprises a viscosity modifier. In one embodiment, the viscosity modifier is an amino acid. In one embodiment, the viscosity modifier is L-proline. In certain embodiments, the viscosity modifier is at a concentration of from 1%+0.2% to 5%+1% w/v. In one embodiment, the viscosity modifier is proline at a concentration of 1.5%+0.3% or about 1.5%. In one embodiment, the viscosity modifier is proline at a concentration of 3%+0.6%, or about 3%.

In a first aspect, the present disclosure provides a novel liquid pharmaceutical formulation, comprising:
a) an anti-GM-CSF antibody of a concentration of 20 mg/ml-200 mg/ml as the antibody;
b) acetate or histidine in a concentration of 10 mM-30 mM as the buffering agent;
c) sucrose or trehalose in a concentration of 130 mM-250 mM as the isotonicity modifier;
d) Polysorbate 80 or Polysorbate 20 in a concentration of 0.01 (w/v) %-0.03 (w/v) %, as surfactant;
   wherein the formulation has a pH of about 4.5 to about 7.5, preferably, about 5.5 to about 6.1.

In a further embodiment, wherein the formulation does not comprise further excipient.

In a preferred embodiment, the present disclosure provides a novel liquid pharmaceutical formulation, comprising:
a) anti-GM-CSF antibody of a concentration of 50 mg/ml-150 mg/ml;
b) histidine in a concentration of 10 mM-20 mM;
c) sucrose in a concentration of 200 mM-220 mM;
d) Polysorbate 80 in a concentration of 0.01 (w/v) %-0.03 (w/v) %;
   wherein the formulation has a pH of 5.5 to 6.1.

In a further embodiment, wherein the formulation does not comprise further excipient.

In a more preferred embodiment, the present disclosure provides a novel liquid pharmaceutical formulation, comprising:
a) anti-GM-CSF antibody of a concentration of 100 mg/ml, wherein the anti-GM-CSF antibody comprises a heavy chain variable region CDR1 (HCDR1) of SEQ ID NO: 1, a HCDR2 of SEQ ID NO: 2, a HCDR3 of SEQ ID NO: 3, a light chain variable region CDR1 (LCDR1) of SEQ ID NO: 4, a LCDR2 of SEQ ID NO: 5, and a LCDR3 of SEQ ID NO: 6;
b) histidine in a concentration of 20 mM;
c) sucrose in a concentration of 220 mM;
d) Polysorbate 80 in a concentration of 0.02 (w/v) %;
   wherein the formulation has a pH of 5.8.

In a further embodiment, wherein the formulation does not comprise further excipient.

The pharmaceutical formulations can be administered to a patient by parenteral routes such as injection (e.g., subcutaneous, intravenous, intramuscular, intraperitoneal, etc.) or percutaneous, mucosal, nasal, pulmonary or oral administration. Numerous reusable pen or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany). Examples of disposable pen or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park, Ill.).

The use of a microinfusor to deliver the pharmaceutical formulations of the present disclosure is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) or viscous solutions.

In certain embodiments, the present disclosure provides a prefilled syringe comprising any of the liquid formulations described herein. In certain embodiments, the syringe is a 1 mL or 2.25 mL long glass syringe filled with a 27-gauge thin wall needle, a fluorocarbon coated rubber plunger and a rubber needle shield.

In one aspect, the liquid pharmaceutical formulations of the present disclosure can be kept at room temperature, refrigerated (e.g., 2-8° C.), or frozen (e.g., −20° C. or −70° C.) for storage.

In certain embodiments, the formulation of any of the preceding aspects has an attribute selected from the group consisting of: (i) the formulation is stable to long-term storage at 50° C., 40° C., 25° C., 5° C., −20° C., −30° C., and −80° C.; (ii) the formulation is low-viscosity (viscosity less than 10 cP); (iv) the formulation is iso-osmolar to physiologic conditions; (v) the formulation is stable to and compatible with intravenous or subcutaneous delivery devices and procedures; and (vi) the formulation is stable to long-term storage in a glass vial or in a prefilled syringe.

In one aspect, the formulation substantially retains its physical and chemical stability, as well as its biological activity upon storage. The storage period is generally selected based on the intended shelf-life of the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period. For example, the liquid formulation is stable at about 40° C. for at least about 3 days, 5 days, 1 week, 2 weeks, 3 weeks, 4 weeks, 5 weeks or 6 weeks. The liquid formulation in another aspect is stable at about 5° C. and/or 25° C. for at least about 1 month, at least about 3 months, at least about 6 months, at least about 9 months, at least about 12 months, at least about 18 months, at least about 24 months, at least about 30 months, or at least about 36 months; and/or stable at about −20° C. and/or −70° C. for at least about 1 month, at least about 3 months, at least about 6 months, at least about 9 months, at least about 12 months, at least about 18 months, at least about 24 months, at least about 30 months, at least about 36 months, at least about 42 months, or at least about 48 months. Furthermore, the liquid formulation may, in some embodiments, be stable following freezing (to, e.g., −80° C.) and thawing, for example following 1, 2 or 3 cycles of freezing and thawing.

The stability of a liquid formulation can be evaluated qualitatively and/or quantitatively in a variety of different ways, including evaluation of dimer, multimer and/or aggregate formation (for example using size exclusion chromatography (SEC), matrix-assisted laser desorption-ionization time-of-flight mass spectrometry (MALDI-TOF MS), analytical ultracentrifugation, light scattering (photon correlation spectroscopy, dynamic light scattering (DLS), static light scattering, multi-angle laser light scattering (MALLS)), flow-based microscopic imaging, electronic impedance (coulter) counting, light obscuration or other liquid particle counting system, by measuring turbidity, and/or by visual inspection): by assessing charge heterogeneity using cation exchange chromatography (CEX), isoelectric focusing (IEF), e.g. capillary technique (cIEF), or capillary zone electrophoresis; amino-terminal or carboxy-terminal sequence analysis: mass spectrometric analysis; SDS-PAGE or SEC analysis to compare fragmented, intact and multimeric (i.e., dimeric, trimeric, etc.) antibody; peptide map (for example tryptic or LYS-C) analysis: evaluating biological activity or antigen binding function of the antibody; and the like. Stability of a solid-state formulation can also be evaluated qualitatively and/or quantitatively in a variety of different ways, including direct tests, such as identifying crystal structure by X-Ray Powder Diffraction (XRPD); evaluating antibody structure in the solid state using Fourier Transform Infrared Spectroscopy (FTIR); and measuring thermal transitions in the lyophilized solid (melting, glass transition, etc.) using Differential Scanning calorimetry (DSC) and indirect tests such as measuring moisture content by Karl Fisher test, e.g., to extrapolate the likelihood of chemical instability through hydrolysis. Instability may involve any one or more of: aggregation (e.g., non-covalent soluble aggregation, covalent soluble aggregation (e.g., disulfide bond rearrangement/scrambling), insoluble aggregation), deamidation (e.g. Asn deamidation), oxidation (e.g. Met oxidation), isomerization (e.g. Asp isomeriation), clipping/hydrolysis/fragmentation (e.g. hinge region fragmentation), succinimide formation, N-terminal extension, C-terminal processing, glycosylation differences, and the like.

### Therapeutic Uses of the Pharmaceutical Formulations

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

In one aspect, the present disclosure provides a method of treating an inflammatory or autoimmune disease or condition in a patient in need thereof, comprising administering to the patient the liquid pharmaceutical formulation provided herein.

In certain embodiments, the inflammatory disease or condition is selected from the group consisting of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, ulcerative colitis and COVID-19.

In certain embodiments, the autoimmune disease or condition is selected from the group consisting of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopeni purpura, myasthenia autoimmune gravis, myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### EXAMPLES

### Example 1. Preparation of pH and Buffer System

155 ml of purified anti-GM-CSF antibody (also called “protein” in all the following experiments, comprising heavy/light chain variable region sequences of SEQ ID NO: 14 and 22 with a Fc region of human IgG1) at a concentration of 24.20 mg/ml was taken and divided into 5 portions, i.e., 3 portions of 17.22 ml and 2 portions of 49 ml; (2) each portion was dialyzed separately into buffer solutions 1-5 (see Table 4-1), after dialysis was completed, buffer solution 4 was equally divided into three portions, two portions among which were adjusted to pH 6.0 and pH 6.5, respectively, with 10 mM of histidine hydrochloride buffer (pH 8.14), and buffer solution 5 was equally divided into three portions, two portions among which were adjusted to pH 7.0 and pH 7.5, respectively, with 20 mmol/L sodium dihydrogen phosphate solution (pH 9.44). 9 portions of protein solutions were then concentrated until the final protein concentration was 50 mg/ml, and the concentrated products were prepared into the following samples (see Table 4-2):

TABLE 4-1

| Formula | | Preparation |
|---|---|---|
| number | Solution formula | volume (L) |
| 1 | 20 mmol/L Sodium acetate-acetic acid pH 4.5 | 1 |
| 2 | 20 mmol/L Sodium acetate-acetic acid pH 5.0 | 1 |
| 3 | 20 mmol/L Sodium acetate-acetic acid pH 5.5 | 1 |
| 4 | 10 mmol/L Histidine hydrochloride-histidine | 3 |
| | pH 5.5 | |
| 5 | 20 mmol/L Sodium dihydrogen phosphate- | 3 |
| | disodium hydrogen phosphate pH 6.5 | |

TABLE 4-2

| Sample batch number | Buffer solution |
|---|---|
| 1 | pH 4.5 20 mmol/L Sodium acetate |
| 2 | pH 5.0 20 mmol/L Sodium acetate |
| 3 | pH 5.5 20 mmol/L Sodium acetate |
| 4 | pH 5.5 10 mmol/L Histidine hydrochloride |
| 5 | pH 6.0 10 mmol/L Histidine hydrochloride |
| 6 | pH 6.5 10 mmol/L Histidine hydrochloride |
| 7 | pH 6.5 20 mmol/L Sodium dihydrogen phosphate |
| 8 | pH 7.0 20 mmol/L Sodium dihydrogen phosphate |
| 9 | pH 7.5 20 mmol/L Sodium dihydrogen phosphate |

The samples were subpackaged (1 ml/tube), wherein one tube of the samples was used for detection at 0 h, and the other samples were placed at 40° C.±2° C., and sampled for detection at 1, 2 and 4 weeks; DSC (at 0 h), appearance inspection, protein concentration detection, purity determination by SEC, purity determination by CE-SDS, and CEX-HPLC were performed (see Table 5-1 and Table 5-2 below).

TABLE 5-1
DSC at 0 h

| Sample batch number | Tml (° C.) | Tm2 (° C.) |
|---|---|---|
| 1 | 62.71718 | 87.84549 |
| 2 | 65.04998 | 86.6765 |
| 3 | 58.29477 | 84.0734 |
| 4 | 65.40558 | 87.88601 |
| 5 | 58.50304 | 83.045 |
| 6 | 59.48022 | 83.4227 |
| 7 | 61.27721 | 83.49064 |
| 8 | 61.61108 | 82.73536 |
| 9 | 61.58672 | 82.70817 |

It can be seen from the DSC results that the Tm values of samples in buffer solutions 1-9 were acceptable. By comparing the results of samples 1-9, the Tm values was in the following relatively decreasing order: 4>2>1>7>8=9>3>5>6. (>represents being superior, and =represents being equivalent).

TABLE 5-2
Appearance inspection at 0 h, 1 week, 2 weeks and 4 weeks

| Sample | | | | | | | | |
|---|---|---|---|---|---|---|---|---|
| batch | Color | | | | Clarity¹ (NTU) | | | |
| number | 0 h | 1 week | 2 weeks | 4 weeks | 0 h | 1 week | 2 weeks | 4 weeks |
| 1 | Colorless | Colorless | Colorless | Colorless | Few | 0.02 | 0.02 | 0.02 |
| | | | | | particles | | | |
| 2 | Colorless | Colorless | Colorless | Colorless | Clear | 0.02 | 0.02 | 0.02 |
| 3 | Colorless | Colorless | Colorless | Colorless | Clear | 0.02 | 0.02 | 0.85 |
| 4 | Colorless | Colorless | Colorless | Colorless | Clear | 0.02 | 0.02 | 0.02 |
| 5 | Colorless | Colorless | Colorless | Colorless | Clear | 0.02 | 0.02 | 0.85 |
| 6 | Colorless | Colorless | Colorless | Colorless | Clear | 0.02-20² | 0.02 | 14.65 |
| 7 | Whitening | / | / | / | No obvious | / | / | / |
| | | | | | particles | | | |
| 8 | Whitening | / | / | / | Few | / | / | / |
| | | | | | particles | | | |
| 9 | Whitening | / | / | / | Few | / | / | / |
| | | | | | particles | | | |

Notes:
¹A visual method was used for clarity test, and an approximate value was reported as the result. There was no reference when detecting at 0 h.
²For the clarity test, only the turbidity standards, 0.02 NTU and 20 NTU, were used for reference, and on intermediate value of turbidity was used.
“/” represents that the test was terminated according to the appearance of being turbid and whitening at 0 h, and the following other detection items were also terminated.

By comparing the remaining samples 1-6, the appearance results were in the following relatively decreasing order: 4=2=1>5=3>6. (>represents being superior, and =represents being equivalent), all results were within the acceptable range (≤6 NTU).

TABLE 5-3
Detection results of protein concentration
at 0 h, 1 week, 2 weeks and 4 weeks

| | |
|---|---|
| Sample | |
| batch | Protein concentration (mg/ml) |

| number | 0 h | 1 week | 2 weeks | 4 weeks |
|---|---|---|---|---|
| 1 | 48.24 | 51.39 | 49.74 | 49.63 |
| 2 | 46.56 | 50.78 | 48.77 | 48.97 |
| 3 | 47.77 | 49.88 | 49.76 | 48.04 |
| 4 | 51.82 | 54.82 | 49.93 | 50.22 |
| 5 | 49.21 | 50.91 | 49.39 | 48.32 |
| 6 | 48.13 | 49.07 | 49.29 | 47.33 |

No obvious change in the protein concentrations of samples 1-6 was found.

TABLE 5-4
Purity determination by SEC at 0 h, 1 week, 2 weeks and 4 weeks

| | | |
|---|---|---|
| Sample | Purity by SEC | Δ |
| batch | (main peak %) | Change |

| number | 0 h | 1 week | 2 weeks | 4 weeks | (%) |
|---|---|---|---|---|---|
| 1 | 99.2 | 98.4 | 98 | 97.7 | −1.5 |
| 2 | 99.1 | 98.2 | 97.5 | 98 | −1.1 |
| 3 | 99.2 | 97.2 | 97 | 96 | −3.2 |
| 4 | 99.0 | 98.2 | 97.7 | 97.9 | −1.1 |
| 5 | 99.0 | 97.9 | 97.4 | 96.7 | −2.3 |
| 6 | 98.6 | 97.5 | 96.7 | 91.8 | −6.8 |

Notes:
“Δ Change” represents the difference value between the detection result at 4 weeks and the detection result at 0 h.

According to changes in purity by SEC, the reduction magnitude was in the following increasing order: 4=2<1<5<3<6 (the final value of sample 6 was less than 95%).

TABLE 5-5
Results of CEX-HPLC at 0 h, 1 week, 2 weeks and 4 weeks

| Sample | | | | | | Δ Change |
|---|---|---|---|---|---|---|
| batch | Electrical | CEX-HPLC (%) | | | | of main |
| number | property | 0 h | 1 week | 2 weeks | 4 weeks | peak (%) |
| 1 | Acid peak | 40.8 | 42.9 | 44.9 | 49 | −9.4 |
| | Main peak | 47.5 | 44.9 | 41.5 | 38.1 | |
| | Basic peak | 11.7 | 12.2 | 13.6 | 13 | |
| 2 | Acid peak | 42.1 | 43.1 | 47.2 | 51 | −7.2 |
| | Main peak | 46.9 | 45.4 | 42.7 | 39.7 | |
| | Basic peak | 11 | 11.5 | 10.1 | 9.3 | |
| 3 | Acid peak | 42.2 | 44.3 | 46.7 | 52 | −5.6 |
| | Main peak | 47.1 | 45.9 | 48.3 | 41.5 | |
| | Basic peak | 11.7 | 9.7 | 4.9 | 6.4 | |
| 4 | Acid peak | 42.4 | 42.9 | 46.5 | 49.5 | −6.6 |
| | Main peak | 48.2 | 52 | 45.2 | 41.6 | |
| | Basic peak | 9.3 | 5.1 | 8.4 | 8.9 | |
| 5 | Acid peak | 42.7 | 44.6 | 47 | 50.2 | −6.6 |
| | Main peak | 48.3 | 46.4 | 46.4 | 41.7 | |
| | Basic peak | 9 | 8.9 | 6.6 | 8.1 | |
| 6 | Acid peak | 41.4 | 45.9 | 49.1 | 54.3 | −3.2 |
| | Main peak | 42.6 | 45.4 | 42.6 | 39.4 | |
| | Basic peak | 16 | 8.7 | 8.3 | 6.3 | |

Notes:
“Δ Change” represents the difference value between the detection result at 4 weeks and the detection result at 0 h.

According to changes in main peak by CEX-HPLC, the reduction magnitude was in the following increasing order: 6<3=4=5=2<1 (all changes in the results were within the acceptable range).

TABLE 5-6
Purity determination by CE-SDS at
0 h, 1 week, 2 weeks and 4 weeks

| Sample | | | | | | Δ |
|---|---|---|---|---|---|---|
| batch | Type of | Purity by CE-SDS (%) | | | | Change |
| number | CE-SDS | 0 h | 1 week | 2 weeks | 4 weeks | (%) |
| 1 | Non-reduced | 95.5 | 94.2 | 89.7 | 87.4 | −8.1 |
| 2 | (main peak) | 95.3 | 94.5 | 92.1 | 91.6 | −3.7 |
| 3 | | 95.2 | 94.3 | 90.9 | 91.2 | −4.0 |
| 4 | | 95.4 | 94.7 | 89.8 | 89.9 | −5.5 |
| 5 | | 95.4 | 94.5 | 92.8 | 90.7 | −4.7 |
| 6 | | 95.5 | 94.2 | 91 | 88.9 | −6.6 |
| 1 | Reduced | 99 | 97.3 | 97.1 | 92.6 | −6.4 |
| 2 | (HC + LC) | 99 | 98 | 97.5 | 94.7 | −4.3 |
| 3 | | 99.3 | 98.4 | 98.3 | 97.5 | −1.8 |
| 4 | | 99 | 98.3 | 98.1 | 95.7 | −3.3 |
| 5 | | 99 | 98.5 | 98.4 | 97.6 | −1.4 |
| 6 | | 99.3 | 98 | 98.2 | 97.3 | −2.0 |

Notes:
“Δ Change” represents the difference value between the detection result at 4 weeks and the detection result at 0 h.

According to changes in purity by non-reduced CE-SDS, the reduction magnitude was in the following increasing order: 2=3=5<4<6<1

According to changes in purity by reduced CE-SDS, the reduction magnitude was in the following increasing order: 5<3=6<4<2<1 (the values of samples 1 and 2 were less than 95%)

TABLE 6
Summary of changes in stability results at 4 weeks

| Detection items | Results | Notes |
|---|---|---|
| Appearance | 4 = 2 = 1 > 5 = 3 > 6 | All appearance results |
| | | were acceptable |
| Protein | 1 = 2 = 3 = 4 = 5 = 6 | No obvious change |
| concentration | | |
| SEC | Δ4 = 2 < 1 < 5 < 3 < 6 | The final value of |
| | | sample 6 was less |
| | | than 95% |
| Reduced | Δ5 < 3 = 6 < 4 < 2 < 1 | The final values of |
| CE-SDS | | samples 1 and 2 were |
| | | less than 95% |
| Non-reduced | Δ2 = 3 = 5 < 4 < 6 < 1 | / |
| CE-SDS | | |
| CEX-HPLC | Δ6 < 3 = 4 = 5 = 2 < 1 | All changes in the |
| | | results were within |
| | | the acceptable range |

Notes:
Appearance results were in the relatively decreasing order, and Δ represents the difference value of results changes.

### Example 2. Screening Composition of Excipients

TABLE 7-1
Solution Preparation

| Buffer | | Preparation |
|---|---|---|
| solution | Solution formula | volume (ml) |
| 1 | 20 mmol/L Histidine hydrochloride | 3000 |
| | pH 5.8 | |
| 2 | 20 mmol/L Histidine hydrochloride, | 100 |
| | 650 mmol/L Sucrose, pH 5.8 | |
| 3 | 20 mmol/L Histidine hydrochloride, | 100 |
| | 650 mmol/L Trehalose, pH 5.8 | |
| 4 | 20 mmol/L Histidine hydrochloride, | 100 |
| | 650 mmol/L Mannitol, pH 5.8 | |
| 5 | 20 mmol/L Histidine hydrochloride, | 50 |
| | 1.3 mol/L Proline, pH 5.8 | |
| 6 | 20 mmol/L Histidine hydrochloride, | 50 |
| | 750 mmol/L Arginine hydrochloride, | |
| | pH 5.8 | |
| 7 | 20 mmol/L Histidine hydrochloride, | 50 |
| | 1.3 mol/L Glycine, pH 5.8 | |
| 8 | 20 mmol/L Histidine hydrochloride, | 50 |
| | 750 mmol/L Sodium chloride, pH 5.8 | |
| 9 | 20 mmol/L Histidine hydrochloride, | 50 |
| | 0.1% Polysorbate 20, pH 5.8 | |
| 10 | 20 mmol/L Histidine hydrochloride, | 50 |
| | 0.1% Polysorbate 80, pH 5.8 | |

306.68 ml of purified protein at a concentration of 8.627 mg/ml was taken. The protein was dialyzed into buffer solution 1 in Table 7-1, and concentrated to 30 ml with a concentration of 84.34 mg/ml. Then 10 ml of buffer solution 1 was added, same was equally divided into 9 portions, 4.4 ml for each portion. To each portion was added 1.1 ml of buffer solutions 2, 3, 4, 5, 6, 7, 8, 9 and 10 in Table 7-1, respectively, to prepare the following samples in Table 7-2:

TABLE 7-2

| Sample | |
|---|---|
| batch | |
| number | Formula |
| 1 | pH 5.8 20 mmol/L Histidine hydrochloride, 130 mmol/L |
| | Sucrose |
| 2 | pH 5.8 20 mmol/L Histidine hydrochloride, 130 mmol/L |
| | Trehalose |
| 3 | pH 5.8 20 mmol/L Histidine hydrochloride, 130 mmol/L |
| | Mannitol |
| 4 | pH 5.8 20 mmol/L Histidine hydrochloride, 260 mmol/L |
| | Proline |
| 5 | pH 5.8 20 mmol/L Histidine hydrochloride, 150 mmol/L |
| | Arginine hydrochloride |
| 6 | pH 5.8 20 mmol/L Histidine hydrochloride, 260 mmol/L |
| | Glycine |
| 7 | pH 5.8 20 mmol/L Histidine hydrochloride, 150 mmol/L |
| | Sodium chloride |
| 8 | pH 5.8 20 mmol/L Histidine hydrochloride, 0.02% |
| | Polysorbate 20 |
| 9 | pH 5.8 20 mmol/L Histidine hydrochloride, 0.02% |
| | Polysorbate 80 |

The stability of each sample was examined based on results of DSC (at 0 h), appearance inspection, protein concentration detection, purity determination by SEC and purity determination by CE-SDS after being placed at 50° C. for 1 week.

TABLE 8-1
DSC results at 0 h

| Sample | | |
|---|---|---|
| batch | Tml | Tm2 |
| number | (° C.) | (° C.) |
| 1 | 67.58782 | 88.66859 |
| 2 | 67.36669 | 88.70947 |
| 3 | 67.49409 | 88.79385 |
| 4 | 67.09545 | 88.70656 |
| 5 | 66.33763 | 87.62923 |
| 6 | 67.98974 | 89.16090 |
| 7 | 64.99342 | 86.43304 |
| 8 | 66.05772 | 88.20136 |
| 9 | 67.11712 | 88.4187 |

The Tm values of plonmarlimab in excipient solutions 1-9 were acceptable.

TABLE 8-2
Appearance inspection results at 0 h, 2 days, 4 days and 1 week

| Sample batch | Color | | | | Clarity (NTU) | | | |
|---|---|---|---|---|---|---|---|---|
| number | 0 h | 48 h | 96 h | 1 week | 0 h | 48 h | 96 h | 1 week |
| 1 | Colorless | Colorless | Colorless | Colorless | 0.85 | 0.02 | 1.78 | 1.78 |
| 2 | Colorless | Colorless | Colorless | Colorless | 0.02 | 0.02 | 1.78 | 1.78 |
| 3 | Colorless | Colorless | Colorless | Colorless | 1.78 | 1.78 | 1.78 | 1.78 |
| 4 | Colorless | Colorless | Colorless | Colorless | 0.85 | 1.78 | 1.78 | 0.85 |
| 5 | Colorless | Colorless | Colorless | Colorless | 4.65 | 4.65 | 4.65 | 4.65 |
| 6 | Colorless | Colorless | Colorless | Colorless | 1.78 | 4.65 | 1.78 | 1.78 |
| 7 | Colorless | Colorless | Colorless | Colorless | 9.45 | 9.45 | 9.45 | 9.45 |
| 8 | Colorless | Colorless | Colorless | Colorless | 1.78 | 1.78 | 1.78 | 1.78 |
| 9 | Colorless | Colorless | Colorless | Colorless | 1.78 | 4.65 | 1.78 | 1.78 |

Notes:
A visual method was used for clarity test, and an approximate value was reported as the result.

It can be seen from the appearance inspection results that the appearance of formula samples 1-9 was within the acceptable range after being placed at 50° C. for 1 week, and the appearance results were in the following relatively decreasing order: 1=2=3=4=6=8=9>5>7.

TABLE 8-3
Detection results of concentration at 0 h, 96 h and 1 week

| Sample | | | |
|---|---|---|---|
| batch | Protein concentration (mg/ml) | | |
| number | 0 h | 96 h | 1 week |
| 1 | 48.02 | 48.01 | 47.76 |
| 2 | 46.24 | 48.46 | 46.46 |
| 3 | 47.77 | 47.17 | 48.56 |
| 4 | 47.90 | 48.02 | 48.26 |
| 5 | 47.46 | 48.17 | 48.68 |
| 6 | 48.01 | 48.79 | 48.68 |
| 7 | 47.17 | 48.63 | 47.74 |
| 8 | 50.87 | 51.51 | 51.53 |
| 9 | 51.41 | 52.57 | 51.45 |

No obvious change in the protein concentrations of formulae 1-9 was found after being placed at 50° C. for 1 week.

TABLE 8-4
Purity determination results by SEC at 0 h and 1 week

| | | |
|---|---|---|
| Sample | Purity by SEC | Δ |
| batch | (main peak %) | Change |

| number | 0 h | 1 week | (%) |
|---|---|---|---|
| 1 | 99.1 | 98.5 | −0.6 |
| 2 | 99.2 | 98.8 | −0.4 |
| 3 | 99.2 | 98.4 | −0.8 |
| 4 | 99.3 | 99.1 | −0.2 |
| 5 | 99.3 | 98.8 | −0.5 |
| 6 | 99.8 | 98.5 | −1.3 |
| 7 | 99.3 | 98.1 | −1.2 |
| 8 | 99.3 | 98.2 | −1.1 |
| 9 | 99.2 | 97.9 | −1.3 |

Purities by SEC of formulae 1-9 were >97% after being placed at 50° C. for 1 week, and the changes in the results were within the acceptable range.

TABLE 8-5
Results of CEX-HPLC at 0 h and 1 week

| | | | |
|---|---|---|---|
| Sample | | | Δ Change |
| batch | Electrical | CEX-HPLC (%) | in main |

| number | property | 0 h | 1 week | peak (%) |
|---|---|---|---|---|
| 1 | Acid peak | 39.7 | 48.2 | −8.1 |
| | Main peak | 51.2 | 43.1 | |
| | Basic peak | 9.1 | 8.7 | |
| 2 | Acid peak | 43.6 | 48.2 | −5.8 |
| | Main peak | 48.5 | 42.7 | |
| | Basic peak | 7.9 | 9.1 | |
| 3 | Acid peak | 47.0 | 48.2 | −2.7 |
| | Main peak | 45.1 | 42.4 | |
| | Basic peak | 7.8 | 9.4 | |
| 4 | Acid peak | 43.9 | 48.2 | −6.8 |
| | Main peak | 47.8 | 41.0 | |
| | Basic peak | 8.3 | 10.7 | |
| 5 | Acid peak | 44.8 | 45.1 | 1.5 |
| | Main peak | 42.9 | 44.4 | |
| | Basic peak | 12.3 | 10.5 | |
| 6 | Acid peak | 44.1 | 49.0 | −2.3 |
| | Main peak | 47.5 | 45.2 | |
| | Basic peak | 8.3 | 5.8 | |
| 7 | Acid peak | 42.6 | 46.1 | −8.1 |
| | Main peak | 51.9 | 43.8 | |
| | Basic peak | 5.5 | 10.1 | |
| 8 | Acid peak | 43.4 | 49.1 | −6.6 |
| | Main peak | 48.9 | 42.3 | |
| | Basic peak | 7.8 | 8.6 | |
| 9 | Acid peak | 49.6 | 50.2 | −6.2 |
| | Main peak | 46.4 | 40.2 | |
| | Basic peak | 4.0 | 9.7 | |

There was a large difference in purity by CEX-HPLC of formulae 1-9 at 0 h, i.e., the highest and lowest values differed by 9%; in contrast, the highest and lowest values differed by 4.0% after being placed at 50° C. for 1 week, which was within the acceptable range, and therefore, the result at 0 h was taken as the basis for formula selection. The results at 0 h followed the ranking: 1 (51.9%)=7>2=4=6=8>9>3>5 (42.9%).

TABLE 8-6
Purity determination results by CE-SDS at 0 h and 1 week

| Purity determination results | | | Δ |
|---|---|---|---|
| by CE-SDS at 0 h and 1 week | Purity by CE-SDS (%) | | Change |
| Sample batch number | 0 h | 1 week | (%) |

Non-reduced CE-SDS: Main peak

| | | | |
|---|---|---|---|
| 1 | 96.0 | 94.5 | −1.5 |
| 2 | 96.5 | 94.5 | −2.0 |
| 3 | 95.9 | 94.4 | −1.5 |
| 4 | 95.8 | 94.2 | −1.6 |
| 5 | 97.2 | 94.3 | −2.9 |
| 6 | 96.3 | 92.9 | −3.4 |
| 7 | 97.1 | 94.5 | −2.6 |
| 8 | 96.1 | 93.9 | −2.2 |
| 9 | 95.9 | 93.0 | −2.9 |

Reduced CE-SDS: HC + LC

| | | | |
|---|---|---|---|
| 1 | 99.2 | 98.1 | −1.1 |
| 2 | 99.1 | 97.7 | −1.4 |
| 3 | 99.2 | 97.3 | −1.9 |
| 4 | 99.2 | 97.6 | −1.6 |
| 5 | 99.6 | 98.3 | −1.3 |
| 6 | 99.2 | 97.9 | −1.3 |
| 7 | 99.5 | 98.3 | −1.2 |
| 8 | 99.2 | 97.2 | −2.0 |
| 9 | 99.2 | 97.7 | −1.5 |

After the samples were placed at 50° C. for 1 week, according to changes in purity by non-reduced CE-SDS, the reduction magnitude was in the following increasing order: 1=3=4<2=7=8<5=6=9, and according to changes in purity by reduced CE-SDS, the reduction magnitude was in the following increasing order: 1=5=6=7=2=9=4<3=8, all results were within the acceptable range.

According to the changes in stability results at 50° C. for 1 week (see Table 9 below), the comparison was carried out for cases with different surfactants, and the difference between polysorbate 80 and polysorbate 20 was not obvious. The comparison was carried out for cases with the remaining excipients, excluding the CEX-HPLC results, the results of samples 1 and 4 were similar and slightly better than the other formulae.

TABLE 9
Summary of changes in stability results at 50° C. at 1 week

| Summary of detection results | | Sample | Type ofexcipient |
|---|---|---|---|
| Detection | Results | 1 | Sucrose |
| items | | 2 | Trehalose |
| | | 3 | Mannitol |
| Appearance | All were within the acceptable range | 4 | Proline |
| | 1 = 2 = 3 = 4 = 6 = 8 = 9 > 5 > 7 | | |
| Protein concentration | There was no significant difference for all | 5 | Arginine hydrochloride |
| SEC | All were within the acceptable range | 6 | Glycine |
| | Δ 1 = 2 = 3 = 4 = 5 < 6 = 7 = 8 = 9 | | |
| CEX-HPLC | There was a significant difference in results at 0 h: | 7 | Sodium Chloride |
| | 1(51.9%) = 7 > 2 = 4 = 6 = 8 > 9 > 3 > 5(42.9%) | | |
| | The difference in results at 1 week was within the acceptable range | | |
| Non-reduced CE-SDS | All were within the acceptable range | 8 | Polysorbate 20 |
| | Δ 1 = 3 = 4 < 2 = 7 = 8 < 5 = 6 = 9 | | |
| Reduced CE-SDS | All were within the acceptable range | 9 | Polysorbate 80 |
| | Δ 1 = 5 = 6 = 7 = 2 = 9 = 4 < 3 = 8 | | |

Notes:
Appearance changes were in the relatively decreasing order, and Δ represents the difference value of results changes.

### Example 3. Study on Protein Solubility

The solubility and viscosity of the protein at concentrations of 100 mg/ml and 150 mg/ml in histidine hydrochloride buffer system (pH 5.8) were investigated so as to confirm that whether the protein samples can be prepared into a product for subcutaneous injection at a high concentration. 3.73 g of the original protein sample was taken, dialyzed into 20 mmol/L histidine hydrochloride (pH 5.5), then the pH was adjusted to 5.8 with 20 mmol/L histidine hydrochloride (pH 6.5), and 84.44 ml, 126.65 ml and 80 ml of the solution were taken, respectively, and concentrated to prepare the following samples:

TABLE 10-1

| Sample number | Target protein concentration |
|---|---|
| 1 | 100 mg/ml |
| 2 | 150 mg/ml |
| 3 | 100 mg/ml |

Notes:
Sample 3 was the same as sample 1 and was used exclusively for clarity test.

Each batch was 2 ml/tube, and there were 6 tubes of samples 1 and 2, respectively, 2 of which were used for detection at 0 h, 2 of which were placed at 4° C. and sampled for detection at 48 h, and 2 of which were placed at 25° C. and sampled for detection at 48 h. Sample 3 was subpackaged into 5 tubes, placed at 25° C. and sampled for clarity test at 48 h. The detection items comprise appearance/color, protein concentration, OD350, viscosity, and clarity (see Tables 10-2 and 10-3).

TABLE 10-2
Detection results

| | Sample 1 | | | Sample 2 | | |
|---|---|---|---|---|---|---|
| | (100 mg/ml) | | | (150 mg/ml) | | |
| | | 48 h | | | 48 h | |
| Detection items | 0 h | 4° C. | 25° C. | 0 h | 4° C. | 25° C. |
| Color | Yellow | Yellow | Yellow | Yellow | Yellow | Yellow |
| Clarity | 0.85 | 0.85 | 0.85 | 0.85 | 0.02 | 0.02 |
| (NTU) | | | | | | |
| Protein concentration | 112.08 | 110.24 | 108.24 | 149.90 | 155.00 | 158.80 |
| (mg/ml) | | | | | | |
| OD350 | 0.4926 | 0.4725 | 0.4912 | 0.6661 | 0.6678 | 0.6770 |
| Viscosity/temperature | 2.98/25.1 | 3.09/25.0 | 3.45/25.3 | 7.38/25.1 | 6.84/25.0 | 6.83/25.0 |
| (cP/° C.) | 3.18/25.2 | 3.03/25.4 | 3.61/25.3 | | 6.29/25.0 | 6.42/25.2 |

Notes:
A visual method was used for clarity test, and an approximate value was reported as the result.

TABLE 10-3
Results of clarity test at 48 h:

| Sample batch number | Temperature | Clarity (NTU) |
|---|---|---|
| Sample 3 | 25° C. | 4.82 |

Notes:
The instrumental method was used for clarity test.

In 20 mmol/L histidine hydrochloride buffer system (pH 5.8), when the protein was at concentrations of 100 mg/ml and 150 mg/ml and the samples were placed at 4° C. and 25° C. for 48 hours, no obvious changes were found in appearance color, clarity, OD350, protein concentration and viscosity, wherein the viscosity was lower than 10 cP and the clarity was lower than 6 NTU. Therefore, the protein of the present disclosure can be developed into a preparation for subcutaneous injection at a high concentration.

### Example 4. Confirmation of Excipient Concentration

An appropriate concentration of sucrose was selected according to the viscosity and osmotic pressure, the durability and stability at pH 5.8 (with the operating range of pH being 5.5-6.1) was investigated, and the concentration of polysorbate 80 in the preparation formula was measured.

TABLE 11-1
Solution preparation

| Solution | | Preparation |
|---|---|---|
| number | Specific components | volume (ml) |
| A | pH 5.8 20 mmol/L Histidine hydrochloride, | 50 |
| | 1250 mmol/L Sucrose | |
| B | pH 5.8 20 mmol/L Histidine hydrochloride, | 50 |
| | 950 mmol/L Sucrose | |
| C | pH 5.8 20 mmol/L Histidine hydrochloride, | 50 |
| | 550 mmol/L Sucrose | |

Three portions of the protein were taken, 480 μl for each portion, to each portion was added 120 μl of solutions A, B and C, respectively, to prepare the following samples as shown in Table 11-2, and the samples were mixed uniformly and then measured for the protein concentration and viscosity, respectively:

TABLE 11-2
Sample solution formula

| Sample | | Target protein |
|---|---|---|
| number | Solution formula | concentration |
| a | pH 5.8 20 mmol/L Histidine hydrochloride, | 100 mg/ml |
| | 250 mmol/L Sucrose | |
| b | pH 5.8 20 mmol/L Histidine hydrochloride, | 100 mg/ml |
| | 190 mmol/L Sucrose | |
| C | pH 5.8 20 mmol/L Histidine hydrochloride, | 100 mg/ml |
| | 110 mmol/L Sucrose | |

TABLE 12
Detection results

| Sample | Protein | Sample viscosity/temperature |
|---|---|---|
| number | concentration (mg/ml) | (cP/° C.) |
| a | 106.25 | 5.27/25.3 |
| b | 104.42 | 4.99/25.2 |
| c | 107.24 | 3.76/25.3 |

As shown in Table 12, there was no difference in protein concentration among the three samples a, b and c, the viscosity of the three samples was acceptable, and the viscosity decreased with the decrease of sucrose concentration.

### Example 5. Experimental Study on Osmotic Pressure

TABLE 13-1
Solution preparation

| Formula | | Preparation |
|---|---|---|
| number | Solution formula | volume (ml) |
| D | pH 5.8 20 mmol/L Histidine hydrochloride, | 50 |
| | 1250 mmol/L Sucrose, 0.1% Polysorbate 80 | |
| E | pH 5.8 20 mmol/L Histidine hydrochloride, | 50 |
| | 1200 mmol/L Sucrose, 0.1% Polysorbate 80 | |
| F | pH 5.8 20 mmol/L Histidine hydrochloride, | 50 |
| | 1150 nimol/L Sucrose, 0.1% Polysorbate 80 | |
| G | pH 5.8 20 mmol/L Histidine hydrochloride, | 10 |
| | 1000 mmol/L Sucrose, 0.1% Polysorbate 80 | |
| H | pH 5.8 20 mmol/L Histidine hydrochloride, | 10 |
| | 950 mmol/L Sucrose, 0.1% Polysorbate 80 | |
| I | pH 5.8 20 mmol/L Histidine hydrochloride, | 10 |
| | 900 mmol/L Sucrose, 0.1% Polysorbate 80 | |

6 portions of the protein were taken, 480 μl for each portion, to each portion was added 120 μl of solutions D, E, F, G, H and I, respectively, to prepare the following samples in Table 13-2:

TABLE 13-2
Formula of sample preparation

| Sample | | Target protein |
|---|---|---|
| number | Solution formula | concentration |
| D | pH 5.8 20 mmol/L Histidine hydrochloride, | 100 mg/ml |
| | 250 mmol/L Sucrose, 0.02% Polysorbate 80 | |
| E | pH 5.8 20 mmol/L Histidine hydrochloride, | 100 mg/ml |
| | 240 mmol/L Sucrose, 0.02% Polysorbate 80 | |
| F | pH 5.8 20 mmol/L Histidine hydrochloride, | 100 mg/ml |
| | 230 mmol/L Sucrose, 0.02% Polysorbate 80 | |
| G | pH 5.8 20 mmol/L Histidine hydrochloride, | 100 mg/ml |
| | 200 mmol/L Sucrose, 0.02% Polysorbate 80 | |
| H | pH 5.8 20 mmol/L Histidine hydrochloride, | 100 mg/ml |
| | 190 mmol/L Sucrose, 0.02% Polysorbate 80 | |
| I | pH 5.8 20 mmol/L Histidine hydrochloride, | 100 mg/ml |
| | 180 mmol/L Sucrose, 0.02% Polysorbate 80 | |

After mixing evenly, the protein concentration and osmotic pressure were detected, respectively, and the D-I formula solution was diluted 5× with 20 mmol/L histidine hydrochloride buffer solution (pH 5.8) and then detected for the osmotic pressure. The detection results were as shown in Table 14 below.

TABLE 14
Detection results of osmotic pressure

| | Protein | Osmotic pressure | Osmotic pressure |
|---|---|---|---|
| Sample | concentration | of buffer solution | of protein solution |
| number | (mg/ml) | (mOsmol/kg) | (mOsmol/kg) |
| D | 103.16 | 312 | 366.7 |
| E | 103.22 | 314 | 365.0 |
| F | 103.92 | 289 | 354.7 |
| G | 98.64 | 259 | 306.7 |
| H | 100.99 | 241 | 278.3 |
| I | 99.42 | 225 | 259 |

### Example 6. Confirmation of pH and Tween Content

TABLE 15-1
Solution preparation

| Formula | | Preparation |
|---|---|---|
| number | Specific components | volume (ml) |
| 1 | pH 5.5 20 mM Histidine hydrochloride, | 50 |
| | 1000 mmol/L Sucrose, 0.1% Polysorbate 80 | |
| 2 | pH 5.8 20 mM Histidine hydrochloride, | 50 |
| | 1000 mmol/L Sucrose, 0.1% Polysorbate 80 | |
| 3 | pH 6.1 20 mM Histidine hydrochloride, | 50 |
| | 1000 mmol/L Sucrose, 0.1% Polysorbate 80 | |
| 4 | pH 5.8 20 mM Histidine hydrochloride, | 50 |
| | 1000 mmol/L Sucrose | |
| 5 | pH 5.8 20 mM Histidine hydrochloride, | 50 |
| | 1000 mmol/L Sucrose, 0.2% Polysorbate 80 | |

3.6 g of protein was dialyzed into 20 mmol/L histidine hydrochloride buffer solution (pH 5.5), divided into three portions with the volumes of 30.47 ml, 103.45 ml and 30.41 ml, respectively, the pH was detected and adjusted to 5.5, 5.8 and 6.1, respectively, then concentrated by using an ultrafiltration centrifuge tube to a final concentration of 125 mg/ml, and the volumes after the concentration were 9.04 ml, 33.26 ml, and 7.95 ml, respectively. 9.6 ml of protein solution at pH 5.8 was taken out, and the other two protein solutions were used, to the three protein solutions were added ¼ volume of buffer solutions 1, 2, and 3, respectively, to prepare samples 1-3 (see Table 15-2) to investigate the effect of different pHs.

The remaining 23.26 ml of protein solution (pH 5.8) in step (1) was equally divided into 3 portions, 7.45 ml for each portion, and to each portion was added 1.86 ml of buffer solutions 4, 2 and 5, respectively, to prepare samples 4-6 (see Table 15-2) to investigate the effect of different concentrations of polysorbate 80.

TABLE 15-2
Formula of sample preparation

| Sample | |
|---|---|
| number | Preparation formula |
| 1 | pH 5.5 20 mM Histidine hydrochloride, |
| | 200 mmol/L Sucrose, 0.02% Polysorbate 80 |
| 2 | pH 5.8 20 mM Histidine hydrochloride, |
| | 200 mmol/L Sucrose, 0.02% Polysorbate 80 |
| 3 | pH 6.1 20 mM Histidine hydrochloride, |
| | 200 mmol/L Sucrose, 0.02% Polysorbate 80 |
| 4 | pH 5.5 20 mM Histidine hydrochloride, |
| | 200 mmol/L Sucrose |
| 5 | pH 5.5 20 mM Histidine hydrochloride, |
| | 200 mmol/L Sucrose, 0.02% Polysorbate 80 |
| 6 | pH 5.5 20 mM Histidine hydrochloride, |
| | 200 mmol/L Sucrose, 0.04% Polysorbate 80 |

Subpackaging: 2 ml/tube for each batch. For samples 1-3, one tube of which was used for detection at 0 h, the other samples were placed at 40° C. and sampled for detection at 1 week, 2 weeks and 4 weeks; for samples 4-6, 2 tubes of which were used for detection at 0 h, and the other samples were placed at 50° C. and sampled for detection at 1 week. Detection items comprise appearance (Table 16-1), protein concentration (Table 16-2), purity by SEC (Table 16-3), purity by CE-SDS (Table 16-4), and purity by CEX-HPLC (Table 16-5).

TABLE 16-1
Results of clarity test

| Sample batch | Storage | Color | | | | Clarity (NTU) | | | |
|---|---|---|---|---|---|---|---|---|---|
| number | condition | 0 h | 1 week | 2 weeks | 4 weeks | 0 h | 1 week | 2 weeks | 4 weeks |
| 1 | 40° C. | Yellow | Yellow | Yellow | Yellow | 4.65 | 4.65 | 4.65 | 4.65 |
| 2 | | Yellow | Yellow | Yellow | Yellow | 4.65 | 4.65 | 4.65 | 4.65 |
| 3 | | Yellow | Yellow | Yellow | Yellow | 4.65 | 4.65 | 4.65 | 4.65 |
| 4 | 50° C. | Yellow | Yellow | / | / | 4.65 | 4.65 | / | / |
| 5 | | Yellow | Yellow | / | / | 4.65 | 4.65 | / | / |
| 6 | | Yellow | Yellow | / | / | 4.65 | 4.65 | / | / |

Notes:
A visual method was used for clarity test, and an approximate value was reported as the result.

TABLE 16-2
Detection results of protein concentration

| Sample | Storage | Protein concentration (mg/ml) | | | |
|---|---|---|---|---|---|
| batch number | condition | 0 h | 1 week | 2 weeks | 4 weeks |
| 1 | 40° C. | 100.68 | 96.69 | 100.16 | 97.45 |
| 2 | | 96.60 | 96.47 | 102.25 | 98.73 |
| 3 | | 101.69 | 98.10 | 104.31 | 97.78 |
| 4 | 50° C. | 98.22 | 94.82 | / | / |
| 5 | | 102.09 | 97.27 | / | / |
| 6 | | 99.24 | 95.67 | / | / |

TABLE 16-3
Results of SEC

| Sample | | | | | | Δ |
|---|---|---|---|---|---|---|
| batch | Storage | SEC (%) | | | | Change |
| number | condition | 0 h | 1 week | 2 weeks | 4 weeks | (%) |
| 1 | 40° C. | 98.3 | 97.0 | 96.7 | 97.1 | −1.2 |
| 2 | | 98.7 | 97.3 | 96.8 | 96.1 | −2.6 |
| 3 | | 98.7 | 97.1 | 96.2 | 96.5 | −2.2 |
| 4 | 50° C. | 99.0 | 96.4 | / | / | −2.6 |
| 5 | | 99.0 | 96.0 | / | / | −3.0 |
| 6 | | 99.0 | 95.5 | / | / | −3.5 |

TABLE 16-4
Results of CEX-HPLC

| Sample | Storage | | CEX-HPLC | | | | Δ |
|---|---|---|---|---|---|---|---|
| batch | condi- | | | 1 | 2 | 4 | Change |
| number | tion | Results | 0 h | week | weeks | weeks | (%) |
| 1 | 40° C. | Acid peak | 41.6 | 43.7 | 46.8 | 50.7 | / |
| | | Main peak | 48.1 | 47.6 | 44.2 | 423 | −5.8 |
| | | Basic peak | 103 | 8.8 | 9 | 7 | / |
| 2 | | Acid peak | 41.9 | 443 | 46 | 50.9 | / |
| | | Main peak | 52.3 | 46.8 | 47.7 | 42.5 | −9.8 |
| | | Basic peak | 5.8 | 9 | 6.3 | 6.6 | / |
| 3 | | Acid peak | 43.1 | 45.5 | 47.6 | 51.3 | / |
| | | Main peak | 50.1 | 44.6 | 45.6 | 42.3 | −7.8 |
| | | Basic peak | 6.8 | 9.9 | 6.8 | 6.4 | / |
| 4 | 50° C. | Acid peak | 43.1 | 48.9 | / | / | / |
| | | Main peak | 45.8 | 40.6 | / | / | −5.2 |
| | | Basic peak | 11.1 | 10.5 | / | / | / |
| 5 | | Acid peak | 43.3 | 50.5 | / | / | / |
| | | Main peak | 45.8 | 39.9 | / | / | −5.9 |
| | | Basic peak | 10.9 | 9.7 | / | / | / |
| 6 | | Acid peak | 42.4 | 50.9 | / | / | / |
| | | Main peak | 50.7 | 36.5 | / | / | −14.2 |
| | | Basic peak | 7 | 12.6 | / | / | / |

TABLE 16-5
Results of CE-SDS

| Sample | | Storage | | | | | Δ |
|---|---|---|---|---|---|---|---|
| batch | CE- | condi- | CE-SDS (%) | | | | Change |
| number | SDS | tion | 0 h | 1 week | 2 weeks | 4 weeks | (%) |
| 1 | NrCE- | 40° C. | 95.9 | 92.0 | 92.9 | 91.4 | −4.5 |
| 2 | SDS | | 95.9 | 92.9 | 93.1 | 90.4 | −5.5 |
| 3 | Main | | 95.1 | 94.0 | 91.9 | 90.3 | −4.8 |
| 4 | peak | 50° C. | 95.5 | 92.6 | / | / | −2.9 |
| 5 | | | 95.2 | 92.6 | / | / | −2.6 |
| 6 | | | 95.2 | 91.8 | / | / | −3.4 |
| 1 | rCE- | 40° C. | 99.3 | 98.8 | 983 | 97.8 | −1.5 |
| 2 | SDS | | 99.3 | 99.1 | 98.1 | 97.1 | −2.2 |
| 3 | | | 99.0 | 99.1 | 98.6 | 97.2 | −1.8 |
| 4 | | 50° C. | 99.2 | 97.4 | / | / | −1.8 |
| 5 | | | 98.5 | 97.2 | / | / | −13 |
| 6 | | | 99.2 | 97.3 | / | / | −1.9 |

By means of comparing the stabilities at 40° C. for 4 weeks, it was found that the pH value fluctuated within the range of 5.5-6.1, there was no obvious change in appearance and protein concentration, and the changes in purity by SEC, CEX-HPLC and CE-SDS were all within the acceptable ranges.

According to comparison of stability at 50° C. for 1 week, it was found that when the concentration of polysorbate 80 was within the range of 0-0.04%, except that the CEX-HPLC results were slightly abnormal (the purity with 0.04% polysorbate 80 decreased faster than that with a low concentration of polysorbate), there was little difference in changes in other results of appearance, protein concentration, purity by SEC, and purity by CE-SDS with different polysorbate 80 contents.

### Example 7. Adjustment of Preparation Buffer Solution

Considering the convenience and controllability of commercial production, the purpose of directly obtaining the target pH was achieved by adjusting the ratio of histidine and histidine hydrochloride in the present disclosure. Moreover, the sucrose (an osmotic pressure regulator) content was also adjusted.

TABLE 17
Preparation of buffer solution

| Formula | | |
|---|---|---|
| number | Formula | Volume (ml) |
| 1 | 6.8 mmol/L Histidine, 13.2 mmol/L Histidine hydrochloride | 50 |
| 2 | 6.8 mmol/L Histidine, 13.2 mmol/L Histidine hydrochloride, | 50 |
| | 200 mmol/L Sucrose | |
| 3 | 7.8 mmol/L Histidine, 12.2 mmol/L Histidine hydrochloride, 200 mmol/L Sucrose | 50 |
| 4 | 8.4 mmol/L Histidine, 11.6 mmol/L Histidine hydrochloride, 210 mmol/L Sucrose | 50 |
| 5 | 9.0 mmol/L Histidine, 11.0 mmol/L Histidine hydrochloride | 50 |
| 6 | 9.0 mmol/L Histidine, 11.0 mmol/L Histidine hydrochloride, | 50 |
| | 200 mmol/L Sucrose | |

The pH of formula solutions 1-6 and the osmotic pressure of formula solutions 3 and 4 were detected

TABLE 18-1
Detection results of pH

| Sample | | |
|---|---|---|
| number | Formula | pH |
| 1 | 6.8 mmol/L Histidine, 13.2 mmol/L Histidine hydrochloride | 5.69 |
| 2 | 6.8 mmol/L Histidine, 13.2 mmol/L Histidine hydrochloride, 200 mmol/L | 5.62 |
| | Sucrose | |
| 3 | 7.8 mmol/L Histidine, 12.2 mmol/L Histidine hydrochloride, 200 mmol/L | 5.76 |
| | Sucrose | |
| 4 | 8.4 mmol/L Histidine, 11.6 mmol/L Histidine hydrochloride | 5.83 |
| | 210 mmol/L Sucrose | |
| 5 | 9.0 mmol/L Histidine, 11.0 mmol/L Histidine hydrochloride | 5.92 |
| 6 | 9.0 mmol/L Histidine, 11.0 mmol/L Histidine hydrochloride, 200 mmol/L | 5.88 |
| | Sucrose | |

TABLE 18-2
Detection results of osmotic pressure

| | | Osmotic | Average osmotic |
|---|---|---|---|
| Sample | | pressure | pressure value |
| number | Formula | (mOsmol/kg) | (mOsmol/kg) |
| 3 | 7.8 mmol/L Histidine | 241/239/240 | 240 |
| | 12.2 mmol/L Histidine | | |
| | hydrochloride | | |
| | 200 mmol/L Sucrose | | |
| 4 | 8.4 mmol/L Histidine | 252/253/254 | 253 |
| | 11.6 mmol/L Histidine | | |
| | hydrochloride | | |
| | 210 mmol/L Sucrose | | |

The osmotic pressure of formula 4 was closer to that of the buffer solution in Table 18-2.

### Example 8. Adjustment of Formula

By means of confirming the pH and measuring the osmotic pressure of batch stock solution through 50 L process development, it was found that, for the scaled-up production, the pH value was higher than the theoretical value, and the osmotic pressure value was lower than the theoretical value, and therefore, the preparation formula was adjusted.

### 8.1 Adjustment of pH and Buffer Concentration

TABLE 19
Solution preparation

| Buffer | | Preparation |
|---|---|---|
| solution | | volume |
| number | Solution formula | (L) |
| 1 | 8.4 mmol/L Histidine, 11.6 mmol/L Histidine | 2 |
| | hydrochloride, 210 mmol/L Sucrose, pH 5.9 | |
| 2 | 7.2 mmol/L Histidine, 12.8 mmol/L Histidine | 2 |
| | hydrochloride, 210 mmol/L Sucrose, pH 5.8 | |
| 3 | 6.2 mmol/L Histidine, 13.8 mmol/L Histidine | 2 |
| | hydrochloride, 210 mmol/L Sucrose, pH 5.7 | |
| 4 | 5.2 mmol/L Histidine, 14.8 mmol/L Histidine | 2 |
| | hydrochloride, 210 mmol/L Sucrose, pH 5.6 | |
| 5 | 4.4 mmol/L Histidine, 15.6 mmol/L Histidine | 2 |
| | hydrochloride, 210 mmol/L Sucrose, pH 5.5 | |

The original protein samples were taken and equally divided into 5 portions, dialyzed with 10 times the volume into the above-mentioned buffer solutions, respectively. The sample batch numbers were Yu-1, Yu-2, Yu-3, Yu-4 and Yu-5, respectively, and the protein concentration and pH of the samples were detected. The dialyzed samples were separately concentrated with ultrafiltration centrifuge tubes to 100 mg/ml with the batch numbers of 1, 2, 3, 4, and 5, and the protein concentration and pH of the samples were detected, respectively.

TABLE 20
Protein concentration and pH of samples

| | Protein concentration | |
|---|---|---|
| Sample batch number | (mg/ml) | pH |
| Yu-1 | 17.32 | 6.05 |
| Yu-2 | 13.33 | 5.88 |
| Yu-3 | 11.73 | 5.80 |
| Yu-4 | 10.70 | 5.68 |
| Yu-5 | 9.20 | 5.59 |
| 1 | 103.33 | 6.04 |
| 2 | 101.37 | 5.88 |
| 3 | 101.73 | 5.79 |
| 4 | 103.92 | 5.68 |
| 5 | 100.45 | 5.57 |

### 8.2 Confirmation of pH

By means of comparing the small-scale preparation development and the 50 L process, the effects of liquid exchange with concentrator tube and ultrafiltration system on pH and osmotic pressure were confirmed.

TABLE 21-1
Solution preparation and corresponding pH, osmotic pressure and conductivity

| Buffer | | | | |
|---|---|---|---|---|
| solution | | | Osmotic pressure | Conductivity |
| number | Solution formula | pH | (mOsmol/kg) | (μs/cm) |
| 1 | 6.2 mmol/L Histidine, 13.8 mmol/L | 5.68 | 258 | 1125 |
| | Histidine hydrochloride, 200 mmol/L | | | |
| | Sucrose | | | |
| 2 | 6.2 mmol/L Histidine, 13.8 mmol/L | 5.69 | 265 | 1113.5 |
| | Histidine hydrochloride, 210 mmol/L | | | |
| | Sucrose | | | |
| 3 | 6.2 mmol/L Histidine, 13.8 mmol/L | 5.68 | 277 | 1084 |
| | Histidine hydrochloride, 220 mmol/L | | | |
| | Sucrose | | | |

150.21 ml of the protein stock solution was taken and divided into three portions, and same were numbered as a, b and c, respectively, and dialyzed into buffer solutions 1-3, respectively, and then concentrated by using an ultrafiltration centrifuge tube to 100 mg/ml. Another portion of the protein stock solution was taken and numbered as d. Same was dialyzed with two 0.005 m² membrane packs (Sartocon Slice Cassette Hydrosart, 30 kD, Sartorius) into buffer solution 2, and concentrated to 100 mg/ml. Detection items comprise protein concentration, osmotic pressure, pH and conductivity (see Table 21-2).

The buffer concentration and sucrose concentration of formula 2 were the same as those of the confirmed batch for process development. Concentration was carried out with the ultrafiltration centrifuge tube to 100 mg/ml, and the osmotic pressure of the sample was higher than that of the confirmed batch sample. In contrast, concentration was carried out with the membrane pack to 100 mg/mL, and the osmotic pressure of the sample was similar to that of the confirmed batch sample and was lower than 300 mOsmol/kg. In other words, the concentration method used in the preparation process of the protein sample of the present disclosure had effects on the osmotic pressure of the concentrated sample. Therefore, in subsequent experiments, the sucrose concentration was adjusted, and samples were prepared with 0.005 m² membrane packs to investigate the osmotic pressure.

TABLE 21-2
Detection results of samples

| | Protein | Osmotic | | |
|---|---|---|---|---|
| Sample | concentration | pressure | | Conductivity through |
| number | (mg/ml) | (mOsmol/kg) | pH | buffer solution (μs/cm) |
| a | 102.12 | 297/294/294 | 5.76 | 941 |
| b | 97.02 | 313/313/316 | 5.72 | 975 |
| c | 104.75 | 322/321/319 | 5.75 | 1012 |
| d | 100.39 | 288/286/287 | 5.81 | / |

In addition, it was found from the detection results of pH that the method for concentrating the samples had no effect on pH, and the pH of samples a-d were all within the range of pH 5.5-pH 6.1.

### Example 9. Confirmation of Sucrose Concentration

The buffer solutions containing 6.2 mmol/L histidine and 13.8 mmol/L histidine hydrochloride were used. By means of comparing different sucrose concentrations, the sucrose concentration in the formula when the osmotic pressure was 300 mOsmol/kg was confirmed, and the preparation formula was confirmed, the stock solution was prepared, and the density of the dialysis buffer solution and the density of the protein stock solution of the present disclosure were detected.

TABLE 22
Solution preparation

| | | Preparation |
|---|---|---|
| Buffer | | volume |
| solution | Formula | (ml) |
| 1 | 6.2 mmol/L Histidine, 13.8 mmol/L Histidine | 1000 |
| | hydrochloride, 225 mmol/L Sucrose | |
| 2 | 6.2 mmol/L Histidine, 13.8 mmol/L Histidine | 1500 |
| | hydrochloride, 220 mmol/L Sucrose | |

The protein solution was dialyzed into buffer solution 1 and concentrated to 100 mg/ml to obtain a stock solution with the batch number of protein stock solution 1, which was detected for protein concentration, osmotic pressure and pH. 8.99 g of protein solution was dialyzed into buffer solution 2 to prepare a stock solution with the batch number of protein stock solution 2, which was detected for protein concentration, osmotic pressure and pH; in addition, the density thereof and the density of the corresponding dialysis buffer solution were measured. The results were as shown in Table 23-1 and Table 23-2 below.

TABLE 23-1
Detection results of protein concentration, pH and osmotic pressure

| | | Protein | | Osmotic | | |
|---|---|---|---|---|---|---|
| Sample | Buffer | concentration | | pressure | | |
| batch number | solution | (mg/ml) | pH | (mOsmol/kg) | | |
| Protein stock | 1 | 100.78 | 5.844 | 308 | 308 | 310 |
| solution 1 | | | | | | |
| Protein stock | 2 | 105.78 | 5.873 | 297 | 300 | 300 |
| solution 2 | | | | | | |

TABLE 23-2
Detection results of density of buffer solution and protein sample

| | | | | | | Average density |
|---|---|---|---|---|---|---|
| Sample | Measured density value (mg/ml) | | | | | value (mg/ml) |
| Buffer solution 2 | 1.0303 | 1.0306 | 1.0304 | 1.0306 | 1.0306 | 1.0305 |
| Protein stock solution 2 | 1.0568 | 1.0566 | 1.0566 | 1.0568 | 10.567 | 1.0567 |

1. A liquid pharmaceutical formulation comprising
(a) 20 mg/ml-200 mg/ml antibody or antigen-binding fragment thereof which binds specifically to human granulocyte macrophage colony-stimulating factor (GM-CSF) protein, wherein the antibody or antigen-binding fragment thereof comprises a heavy chain variable region CDR1
(HCDR1) of SEQ ID NO: 1, a HCDR2 of SEQ ID NO: 2, a HCDR3 of SEQ ID NO: 3, a light chain variable region
CDR1 (LCDR1) of SEQ ID NO: 4, a LCDR2 of SEQ ID NO: 5, and a LCDR3 of SEQ ID NO: 6;
(b) 10 mM-30 mM pH buffer agent;
(c) 130 mM-250 mM isotonicity modifier; and
(d) 0.01% (w/v)-0.03% (w/v) surfactant,
wherein the formulation has a pH of 4.5-7.5.

2. The liquid pharmaceutical formulation of claim 1, wherein the antibody or antigen-binding fragment thereof comprises a heavy chain variable region comprising one or more amino acid residues selected from the group consisting of:
(a) Glu at position 1,
(b) Arg at position 98,
(c) Ser at position 72,
(d) Ala at position 68,
(e) Leu at position 70,
(f) Ile at position 48,
(g) Asp at position 26, and
(h) Leu at position 29,
according to Kabat numbering, and combinations thereof.

3. The liquid pharmaceutical formulation of claim 2, wherein the heavy chain variable region comprises at least (a) Glu at position 1.

4. The liquid pharmaceutical formulation of claim 2, wherein the heavy chain variable region comprises a fragment of DYTLT (SEQ ID NO: 23) or GYTFT (SEQ ID NO: 24) starting at position 26 according to Kabat numbering.

5. The liquid pharmaceutical formulation of claim 1, wherein the antibody or antigen-binding fragment thereof comprises a light chain variable region comprising one or more amino acid residues selected from the group consisting of:
(a) Ala at position 46,
(b) Asp at position 60,
(c) Asp at position 70,
(d) Ser at position 43, and
(e) Phe at position 87,
according to Kabat numbering, and combinations thereof.

6. The liquid pharmaceutical formulation of claim 1, wherein the antibody or antigen-binding fragment thereof comprises a heavy chain variable region comprising an amino acid sequence selected from the group consisting of SEQ ID NO: 8-17.

7. The liquid pharmaceutical formulation of claim 6, wherein the antibody or antigen-binding fragment thereof comprises a light chain variable region comprising an amino acid sequence selected from the group consisting of SEQ ID NO: 19-22.

8. The liquid pharmaceutical formulation of claim 1, wherein the antibody or antigen-binding fragment thereof comprises a heavy chain variable region comprises the amino acid sequence of SEQ ID NO: 14 and a light chain variable region comprises the amino acid sequence of SEQ ID NO: 22.

9. The liquid pharmaceutical formulation of claim 1, wherein the pH buffer agent comprises citric acid, HEPES, PBS, histidine, potassium acetate, potassium citrate, potassium phosphate (KH₂PO₄), sodium acetate, sodium bicarbonate, sodium citrate, sodium phosphate (NaH₂PO₄), Tris base, and Tris-HCl.

10. The liquid pharmaceutical formulation of claim 9, wherein the pH buffer agent comprises about 20 mM histidine.

11. The liquid pharmaceutical formulation of claim 10, wherein the 20 mM histidine comprises about 6.2 mM histidine and about 13.8 mM histidine hydrochloride monohydrate.

12. The liquid pharmaceutical formulation of claim 11, wherein the pH buffer agent comprises about 20 mM NaAc.

13. The liquid pharmaceutical formulation of claim 1, wherein the pH is ranged from about 5.5 to about 6.1.

14. The liquid pharmaceutical formulation of claim 13, wherein the pH is about 5.8.

15. The liquid pharmaceutical formulation of claim 1, wherein the isotonicity modifier is one or more selected from the group consisting of sucrose, trehalose, mannitol, arginine, and sodium chloride.

16. The liquid pharmaceutical formulation of claim 15, wherein the isotonicity modifier comprises from about 200 mM to about 220 mM sucrose.

17. The liquid pharmaceutical formulation of claim 1, wherein the liquid pharmaceutical formulation comprises 50-150 mg/ml of the antibody.

18. The liquid pharmaceutical formulation of claim 1, wherein the liquid pharmaceutical formulation comprises 50-150 mg/ml of the antibody, 10-20 mM histidine, 200-220 mM sucrose, and 0.01-0.03% (w/v) polysorbate 80, and its pH is about 5.5-6.1.

19. The liquid pharmaceutical formulation of claim 18, wherein the liquid pharmaceutical formulation comprises about 100 mg/ml of the antibody, about 20 mM histidine, about 220 mM sucrose, and about 0.02% (w/v) polysorbate 80, and its pH is about 5.8.

20. A method of treating an inflammatory or autoimmune disease or condition in a patient in need thereof, comprising administering to the patient the liquid pharmaceutical formulation of claim 1.


SEQUENCE LISTING (24 sequences)

SEQ ID NO: 1 - protein, 5 aa, synthetic construct
SHYLH

SEQ ID NO: 2 - protein, 17 aa, synthetic construct
WIFPGDDKTKYNEKFKG

SEQ ID NO: 3 - protein, 11 aa, synthetic construct
GTKYLNWNFDV

SEQ ID NO: 4 - protein, 11 aa, synthetic construct
KANQNVGTTLA

SEQ ID NO: 5 - protein, 7 aa, synthetic construct
SASYRYS

SEQ ID NO: 6 - protein, 9 aa, synthetic construct
HQYTTYPLT

SEQ ID NO: 7 - protein, 120 aa, synthetic construct
QVQLQQSGPELVKPGTSMKISCKTSDYTLTSHYLHWVKQRPGQGLEWIGWIFPGDDKTKYNEKFKGKATLTSDKTSNTAYMQLSSLTSEESAVYFCARGTKYLNWNFDVWGTGTTVTVSS

SEQ ID NO: 8 - protein, 120 aa, synthetic construct
EVQLVQSGAEVKKPGATVKISCKVSGYTFTSHYLHWVQQAPGKGLEWMGWIFPGDDKTKYNEKFKGRVTITADTSTDTAYMELSSLRSEDTAVYYCARGTKYLNWNFDVWGQGTTVTVSS

SEQ ID NO: 9 - protein, 120 aa, synthetic construct
EVQLVQSGAEVKKPGATVKISCKVSGYTFTSHYLHWVQQAPGKGLEWMGWIFPGDDKTKYNEKFKGRVTITSDTSTDTAYMELSSLRSEDTAVYYCARGTKYLNWNFDVWGQGTTVTVSS

SEQ ID NO: 10 - protein, 120 aa, synthetic construct
EVQLVQSGAEVKKPGATVKISCKVSGYTFTSHYLHWVQQAPGKGLEWMGWIFPGDDKTKYNEKFKGRATLTSDTSTDTAYMELSSLRSEDTAVYYCARGTKYLNWNFDVWGQGTTVTVSS

SEQ ID NO: 11 - protein, 120 aa, synthetic construct
EVQLVQSGAEVKKPGATVKISCKVSGYTFTSHYLHWVQQAPGKGLEWIGWIFPGDDKTKYNEKFKGRATLTSDTSTDTAYMELSSLRSEDTAVYYCARGTKYLNWNFDVWGQGTTVTVSS

SEQ ID NO: 12 - protein, 120 aa, synthetic construct
EVQLVQSGAEVKKPGATVKISCKVSDYTLTSHYLHWVQQAPGKGLEWIGWIFPGDDKTKYNEKFKGRATLTSDTSTDTAYMELSSLRSEDTAVYYCARGTKYLNWNFDVWGQGTTVTVSS

SEQ ID NO: 13 - protein, 120 aa, synthetic construct
EVQLVQSGAEVKKPGASVKVSCKASGYTFTSHYLHWVRQAPGQGLEWMGWIFPGDDKTKYNEKFKGRVTMTRDTSISTAYMELSRLRSDDTAVYYCARGTKYLNWNFDVWGQGTTVTVSS

SEQ ID NO: 14 - protein, 120 aa, synthetic construct
EVQLVQSGAEVKKPGASVKVSCKASGYTFTSHYLHWVRQAPGQGLEWMGWIFPGDDKTKYNEKFKGRVTMTSDTSISTAYMELSRLRSDDTAVYYCARGTKYLNWNFDVWGQGTTVTVSS

SEQ ID NO: 15 - protein, 120 aa, synthetic construct
EVQLVQSGAEVKKPGASVKVSCKASGYTFTSHYLHWVRQAPGQGLEWMGWIFPGDDKTKYNEKFKGRVTLTSDTSISTAYMELSRLRSDDTAVYYCARGTKYLNWNFDVWGQGTTVTVSS

SEQ ID NO: 16 - protein, 120 aa, synthetic construct
EVQLVQSGAEVKKPGASVKVSCKASGYTFTSHYLHWVRQAPGQGLEWIGWIFPGDDKTKYNEKFKGRATLTSDTSISTAYMELSRLRSDDTAVYYCARGTKYLNWNFDVWGQGTTVTVSS

SEQ ID NO: 17 - protein, 120 aa, synthetic construct
EVQLVQSGAEVKKPGASVKVSCKASDYTLTSHYLHWVRQAPGQGLEWIGWIFPGDDKTKYNEKFKGRATLTSDTSISTAYMELSRLRSDDTAVYYCARGTKYLNWNFDVWGQGTTVTVSS

SEQ ID NO: 18 - protein, 107 aa, synthetic construct
DIVLTQPQKFLSTSVGDRVSVTCKANQNVGTTLAWYQQKPGQSPKALIYSASYRYSGVPDRFTGSGSGTDFTLTISNVQSEDLAEYFCHQYTTYPLTFGGGTKLEIK

SEQ ID NO: 19 - protein, 107 aa, synthetic construct
DIQLTQSPSFLSASVGDRVTITCKANQNVGTTLAWYQQKPGKAPKLLIYSASYRYSGVPSRFSGSGSGTEFTLTISSLQPEDFATYYCHQYTTYPLTFGGGTKVEIK

SEQ ID NO: 20 - protein, 107 aa, synthetic construct
DIQLTQSPSFLSASVGDRVTITCKANQNVGTTLAWYQQKPGKAPKALIYSASYRYSGVPSRFSGSGSGTEFTLTISSLQPEDFATYYCHQYTTYPLTFGGGTKVEIK

SEQ ID NO: 21 - protein, 107 aa, synthetic construct
DIQLTQSPSFLSASVGDRVTITCKANQNVGTTLAWYQQKPGKAPKALIYSASYRYSGVPDRFSGSGSGTDFTLTISSLQPEDFATYYCHQYTTYPLTFGGGTKVEIK

SEQ ID NO: 22 - protein, 107 aa, synthetic construct
DIQLTQSPSFLSASVGDRVTITCKANQNVGTTLAWYQQKPGKSPKALIYSASYRYSGVPDRFSGSGSGTDFTLTISSLQPEDFATYFCHQYTTYPLTFGGGTKVEIK

SEQ ID NO: 23 - protein, 5 aa, synthetic construct
DYTLT

SEQ ID NO: 24 - protein, 5 aa, synthetic construct
GYTFT